SEQUENCE LISTING (166 sequences)

SEQ ID NO: 1 - DNA, 1140 bp, Bacillus licheniformis
  cds aprE
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtatacagcacttacccaacgaacacttatgcaacattgaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaataa

SEQ ID NO: 2 - protein, 379 aa, Bacillus licheniformis
  subtilisin carsberg protease
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 3 - DNA, 1146 bp, Bacillus subtilis
  cds aprE
gtgagaagcaaaaaattgtggatcagcttgttgtttgcgttaacgttaatctttacgatggcgttcagcaacatgtctgcgcaggctgccggaaaaagcagtacagaaaagaaatacattgtcggatttaaacagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgtaccttctgaaacaaacccataccaggacggcagttctcacggtacgcatgtagccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtcgttgacaaagccgtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgcaggttctgagcttgatgtgatggctcctggcgtgtccatccaaagcacacttcctggaggcacttacggcgcttataacggaacgtccatggcgactcctcacgttgccggagcagcagcgttaattctttctaagcacccgacttggacaaacgcgcaagtccgtgatcgtttagaaagcactgcaacatatcttggaaactctttctactatggaaaagggttaatcaacgtacaagcagctgcacaataa

SEQ ID NO: 4 - protein, 381 aa, Bacillus subtilis
  subtilisin protease
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 5 - DNA, 1146 bp, Bacillus pumilus
  cds aprE1
gtgaaaaagaaaaatgtgatgacaagtgttttattggctgtccctcttctgttttcagcaggatttggaggctccatggcaaatgccgagacggtctcaaagtcagatagtgaaaagagctatattgttggctttaaagcctccgccaccacaaacagctctaagaaacaagccgtcactcaaaatggtggaaaactagaaaagcaatatcgtctcattaatgccgcacaagtaaagatgtcagaacaagccgcgaaaaaactggaacatgaccctagcattgcatacgtagaagaagaccacaaagcagaagcatatgcacaaaccgtcccttatggaatccctcaaatcaaagctccagctgtacacgctcaaggttataaaggtgctaatgtcaaagtagctgtccttgatacaggcatccacgctgcacaccctgacttaaatgttgcaggcggtgccagcttcgtcccttcagagccaaatgccacccaagactttcaatcacatggaactcacgtagccggaacgatcgctgcccttgataacacaattggtgttcttggggtcgctccaagtgcttctctatatgctgttaaagtattagaccgttatggcgacggacaatatagctggatcattagcggtattgaatgggcagtagccaataatatggatgtcatcaacatgagcttaggcggaccaaacggttcaacagcacttaaaaatgccgttgatacagcgaataaccgcggagtcgttgttgtggcagctgcaggtaactcaggttccactggttctacaagtacagttggctatccagcaaaatatgattccaccattgctgttgcgaatgtgaacagcaacaatgtcagaaatacgtcttccagcgcaggtcctgaattagatgtttctgcacctggtacttctattttaagtacagtaccaagcagtggatacacatcttatactggaacatctatggcgtctcctcatgtagcaggagcagcagcgcttattctttctaagtacccgaatctatcaacttctcaggttcgccagcgcttagaaaacactgcaacaccgcttggtagctccttctattacggaaaagggttaatcaacgttcaagcggcttctaactaa

SEQ ID NO: 6 - protein, 381 aa, Bacillus pumilus
  subtilisin protease
MKKKNVMTSVLLAVPLLFSAGFGGSMANAETVSKSDSEKSYIVGFKASATTNSSKKQAVTQNGGKLEKQYRLINAAQVKMSEQAAKKLEHDPSIAYVEEDHKAEAYAQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKNAVDTANNRGVVVVAAAGNSGSTGSTSTVGYPAKYDSTIAVANVNSNNVRNTSSSAGPELDVSAPGTSILSTVPSSGYTSYTGTSMASPHVAGAAALILSKYPNLSTSQVRQRLENTATPLGSSFYYGKGLINVQAASN

SEQ ID NO: 7 - DNA, 434 bp, Bacillus subtilis
  5-prime region to aprE gene comprising promoter aprE
cttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 8 - DNA, 222 bp, Bacillus subtilis
  promoter aprE - 222
gcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 9 - DNA, 585 bp, Bacillus pumilus
  5-prime region to aprE gene comprising promoter aprE
agaacctgttatataaacaggttccagcgtgtagacaaaccttcgcattcgttgtcaggtctgcgcgccggtgctcacgaatgtcaaattcgctccgcgccagtgctcggccttcctagacttcaaaggttttctatcacgctgaaaagaagacaaagtgctaaaataaagatcattttagcactttgtcaacaatctggaacctgttatataaacaggttcttttaaatgacaaaaacaatgataaaataatatttttttatatcgaaattcgaaatagctgctagacgtttctacctattttaaggcttttcgggtatcgaatatttctccgataatggatcataagaaaaatagcatacttcctttttaatagataatcgctgaaacagtagaataaacatattttaccactatttccaagtgacttaattccccaattttcgctaggactttcacaaaaattcaggtctactcttatttgcctacttcccttaaactgaatatacagaataatcaaacgtctcattcttatagactacggatgattattctgaaataagaaaaaagggatgtggattgtgc

SEQ ID NO: 10 - DNA, 357 bp, Bacillus pumilus
  promoter aprE - 357
atgacaaaaacaatgataaaataatatttttttatatcgaaattcgaaatagctgctagacgtttctacctattttaaggcttttcgggtatcgaatatttctccgataatggatcataagaaaaatagcatacttcctttttaatagataatcgctgaaacagtagaataaacatattttaccactatttccaagtgacttaattccccaattttcgctaggactttcacaaaaattcaggtctactcttatttgcctacttcccttaaactgaatatacagaataatcaaacgtctcattcttatagactacggatgattattctgaaataagaaaaaagggatgtggattgtgc

SEQ ID NO: 11 - DNA, 381 bp, Bacillus licheniformis
  5-prime region to aprE gene comprising promoter aprE
atctttcacccgtttctgtatgcgatatattgcatattttaatagatgatcgacaaggccgcaacctccttcggcaaaaaatgatctcataaaataaatgaatagtattttcataaaatgaatcagatggagcaatctcctgtcattcgcggccctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagta

SEQ ID NO: 12 - DNA, 299 bp, Bacillus licheniformis
  promoter aprE - 299
gatctcataaaataaatgaatagtattttcataaaatgaatcagatggagcaatctcctgtcattcgcggccctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagat

SEQ ID NO: 13 - DNA, 227 bp, Bacillus licheniformis
  promoter aprE - 227
ctcgggacctctttccctgccaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagat

SEQ ID NO: 14 - DNA, 181 bp, Bacillus licheniformis
  promoter aprE - 181
tcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagat

SEQ ID NO: 15 - DNA, 165 bp, Bacillus licheniformis
  promoter aprE - 165
tctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagat

SEQ ID NO: 16 - DNA, 156 bp, Bacillus licheniformis
  promoter aprE - 156
gttattaataaccaaaaaattttaaattggtcctccaaaaaaataggcctaccatataattcattttttttctataataaattaacagaataattggaatagattatattatccttctatttaaattattctgaataaagaggaggagagtgagat

SEQ ID NO: 17 - DNA, 143 bp, Bacillus licheniformis
  promoter aprE - 143
aaaaaattttaaattggtcctccaaaaaaataggcctaccatataattcattttttttctataataaattaacagaataattggaatagattatattatccttctatttaaattattctgaataaagaggaggagagtgagat

SEQ ID NO: 18 - DNA, 125 bp, Bacillus licheniformis
  promoter aprE - 125
cctccaaaaaaataggcctaccatataattcattttttttctataataaattaacagaataattggaatagattatattatccttctatttaaattattctgaataaagaggaggagagtgagat

SEQ ID NO: 19 - DNA, 200 bp, Bacillus sp.
  promoter aprE ( ACWC01000005 )
tgaagcggtctattcatactttcgaactgaacatttttctaaaacagttattaataaccaaaaattttaaattggtcctccaaaaaaataggcctaccatataattcatttttttctataataaattaacagaataattggaatagattatattatccttctatttaaattattctgaataaagaggaggagagtgagta

SEQ ID NO: 20 - DNA, 200 bp, Bacillus sp.
  promoter aprE ( AHIF01000002 )
caccggaatcgagcgcttgtccccgcagttttctgatcctttccgcaaacggccagtctttatttctttctcctgtgcagatttggccgattacccggttcacgtctcgttctatcgaatcccaaagcgccggcgataccgcttcaatgatacatttgttcagcacgtatagaacccgcctgatatcgatgtgctgaagt

SEQ ID NO: 21 - DNA, 200 bp, Bacillus sp.
  promoter aprE ( AJLW01000021 )
aagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagta

SEQ ID NO: 22 - DNA, 200 bp, Bacillus sp.; identical to SEQ ID NO: 21
  promoter aprE ( CP010524 )

SEQ ID NO: 23 - DNA, 200 bp, Bacillus sp.
  promoter aprE ( CP017247 )
gaagcggtctattcatactttcgaaccgaatatttttctaaaacagttattaataaccaataaatttaaattggccgttcaaaaaaatgggtctaccatataattcattttttttctataataaattaacagaataattggaatagattatattacccttctatttaaattattctgaataaagaggaggagagtgagta

SEQ ID NO: 24 - DNA, 200 bp, Bacillus sp.
  promoter aprE ( CP018249 )
gaagcggtctattcatactttcgaaccgaatatttttctaaaacagttattaataaccaataaatttaaattggccgttcaaaaaaatgggtctaccatataattcattttttttctataataaattaacagaataattggaatagattatattatccttctatttaaattattctgaataaagaggaggagagtgagta

SEQ ID NO: 25 - DNA, 200 bp, Bacillus sp.
  promoter aprE ( AJLV01000026 )
gaagcggtctattcatactttcgaaccgaatatttttctaaaacagttattaataaccaataaatttaaattggccgttcaaaaaaatgggtctaccatataattcattttttttctataataaattaacagaataattagaatagaatatattattcttctatttcaattattctgaataaaacggaggagagtgagta

SEQ ID NO: 26 - DNA, 200 bp, Bacillus sp.
  promoter aprE ( AMWQ01000025 )
gaagcggtctattcatactttcgaaccgaatatttttctaaaacagttattaataaccaataaatttaaattggccgttcaaaaaaatgggtctaccatataattcattttttttctataataaattaacagaataattagaatagagtatattattcttctatttcaattattctgaataaaacggaggagagtgagta

SEQ ID NO: 27 - DNA, 196 bp, Bacillus sp.
  promoter aprE ( AOFM01000003 )
tcttttttcattactttagcatgtgatatttttctaaagtgggaaatgaaacttaaaaaaacaaaaatattttttcaaaaaaataggcctaccatatatttcattttttttctataataaattaacagaataattcgaatagggtttattataactctttttggattattctgaataaaacggaggagagtgagca

SEQ ID NO: 28 - DNA, 200 bp, Bacillus sp.
  promoter aprE ( LT603683 )
tcggctagtttcattcttaagacctttagatttgttctaaaataggaaaagaagctcgaaaaatcaaatatattttttcaaaaaaataggtctaccatatatttcattttttctatataataaattaacagaataattggaatagaagatattatcaccatatttcaattattctgaataaaaatggaggagagtgatta

SEQ ID NO: 29 - DNA, 181 bp, Bacillus sp.
  promoter aprE ( AFSI01000014 )
catgtctattttcgttcttttcatgtatacagttatttcgaagcgagagatgatatacctaaatagaaataaaacaatctcaaaaaaatgggtctactaaaatattatccaatctattacaataaattcacagaatagtctatcaataggctgctctgaaattgtaaaaggagagggtaaa

SEQ ID NO: 30 - DNA, 178 bp, Bacillus sp.
  promoter aprE ( AFSH01000056 )
gtctattttcgttcttttcaagaaattagttatttcgagacgagagatgatatacctaaatagagataaaatcatcgcaaaaaaataggtctactaaaatatgattgcatctattacaataaatttacagaatagtcttttaaaagtctagtctgaatttacaaaaggagagggtaaa

SEQ ID NO: 31 - DNA, 181 bp, Bacillus sp.
  promoter aprE ( APIS01000010 )
tcatgtctattttcgttcttttcatgaatgcagttatttcgaaacgagagatgatatacctaaatagaaataaaacaatctcaaaaaaatgggtctactaaaatagtatccaatctattacaataaattcacagaatagtctattaataggctgctctgaaattgtaaaggagagggtaaa

SEQ ID NO: 32 - DNA, 181 bp, Bacillus sp.; identical to SEQ ID NO: 31
  promoter aprE ( ASJT01000029 )

SEQ ID NO: 33 - DNA, 178 bp, Bacillus sp.
  promoter aprE ( CP003492 )
gtctattttcgtttttttcatgaaaatagttatttcgaagcgagagatgatatacctaaatagagataaaatcatctcaaaaaaatgggtctactaaaatattattccatctattacaataaattcacagaatagtctttttaaagtctactctgaatttttaaaaggagagggtaaa

SEQ ID NO: 34 - DNA, 176 bp, Bacillus sp.
  promoter aprE ( AEHM01000033 )
ctattttcgttcttttcataaaattagttatttcgaagcgagagatgatatacctaaatagagataaaatcatcgcgaaaaaatgggtctactaaaatattattccatctattacaataaattaacagaatagtcttttaaggggctactctgaatctttaaaaggagagggtaaa

SEQ ID NO: 35 - DNA, 175 bp, Bacillus sp.
  promoter aprE ( AMXN01000004 )
tattttcgttcttttcataaaattagttatttcgaagcgagagatgatatacctaaatagagataaaatcatcgcaaaaaaatgggtctactaaaatattattccatctattacaataaattaacagaatagtcccttaaggggctactctgaatctttaaaaggagagggtaaa

SEQ ID NO: 36 - DNA, 175 bp, Bacillus sp.; identical to SEQ ID NO: 35
  promoter aprE ( CP013984 )

SEQ ID NO: 37 - DNA, 178 bp, Bacillus sp.
  promoter aprE ( AEFY01000038 )
gccaattttcattttttctatatttttagatatttcgaaacgagagatgatatacctaaatagaattaaaacgttcgtaaaaaaatgggtctactaaaaaacgtttccatctattacaataaatttacagaataatcccaaagaggattattctaaattcgtataaggagagggtaaa

SEQ ID NO: 38 - DNA, 178 bp, Bacillus sp.
  promoter aprE ( CP011802 )
gccaattttcattttttctatatttttagatctttcgaaacgagagatgatatacctaaatagaattaaaacgctcgtaaaaaaatgggtctactaaaaaacgtttccatctattacaataaatttacagaataatcccaaagaggattattctaaattcgtataaggagagggtaaa

SEQ ID NO: 39 - DNA, 177 bp, Bacillus sp.
  promoter aprE ( AFSG01000015 )
gtctattttcgttcttttcataaaattagtgacttggaagcgagagatgatatacctaaatagagatgaaatcatcgcgaaaaaatgggtctactaatatattattccatctattacaattaattaacagaatagtctttaaaagtctactctgagtttttaaaaggagagggtaaa

SEQ ID NO: 40 - DNA, 178 bp, Bacillus sp.
  promoter aprE ( AP011541 )
gtctattttcgttcttttcatgaaaatagttatttcgaagcgagagatgatatacctaaatagagataaaatcatctcaaaaaaatgggtctactaaaatattattccatctattacaataaatgcacagaatagtcttttaaaagtctactctgaatttttaaaaggagagggtaaa

SEQ ID NO: 41 - DNA, 178 bp, Bacillus sp.
  promoter aprE ( ANIP01000006 )
gtctattttcgttcttttcatgaaaatagttatttcgaagcgagagatgatatacctaaatagagataaaatcatctcaaaaaaatgggtctactaaaatattattccatctattacaataaattcacagaatagtcttttaaaagtctactctgaatttttaaaaggagagggtaaa

SEQ ID NO: 42 - DNA, 178 bp, Bacillus sp.
  promoter aprE ( CP007173 )
gtctattttcgttcttttcacgaaagtagttatttcgaagcgagagatgatatacctaaatagagataaaatcatctcaaaaaaatgggtctactaaaatattattccatctattacaataaattcacagaatagtcttttaaaagtctactctgaatttttaaaaggagagggtaaa

SEQ ID NO: 43 - DNA, 178 bp, Bacillus sp.
  promoter aprE ( CP011534 )
gtctattttcgttcttttcatgaaagtagttatttcgaagcgagagatgatatacctaaatagagataaaatcatctcaaaaaaatgggtctactaaaatattattccatctattacaataaattcacagaatagtcttttaaaagtctactctgaatttttaaaaggagagggtaaa

SEQ ID NO: 44 - DNA, 178 bp, Bacillus sp.; identical to SEQ ID NO: 42
  promoter aprE ( CP004019 )

SEQ ID NO: 45 - DNA, 178 bp, Bacillus sp.
  promoter aprE ( AMCA01000186 )
gtctattttcgttcttttcatgaaaatagttatttcgaagcgagagatgatatacctaaatagagataaaatcatctcaaaaaaatgggtctactaaaatattattccatctattacaataaattcacagaatagtcttttaagagtctactctgaatttttaaaaggagagggtaaa

SEQ ID NO: 46 - DNA, 177 bp, Bacillus sp.
  promoter aprE ( AFSF01000114 )
tctattttcgttcttttcatgaaaatagttatttcgaagcgagagatgatatacctaaatagagataaaatcatctcaaaaaaatgggtctactaaaatattattccatctattacaataaattcacagaatagtcttttaaaagtctactctgaatttttaaaaggagagggtaaa

SEQ ID NO: 47 - DNA, 178 bp, Bacillus sp.; identical to SEQ ID NO: 41
  promoter aprE ( CP017676 )

SEQ ID NO: 48 - DNA, 177 bp, Bacillus sp.
  promoter aprE ( CP015222 )
ctattttcgttcttttcatgaaaatagttatttcgaagcgagagatgatatacctaaatagagataaaatcatctcaaaaaaaatgggtctactaaaatattattccatctattacaataaattcacagaatagtcttttaaaagtctactctgaatttttaaaaggagagggtaaa

SEQ ID NO: 49 - DNA, 177 bp, Bacillus sp.; identical to SEQ ID NO: 46
  promoter aprE ( ABQN01000007 )

SEQ ID NO: 50 - DNA, 178 bp, Bacillus sp.; identical to SEQ ID NO: 41
  promoter aprE ( CP007409 )

SEQ ID NO: 51 - DNA, 178 bp, Bacillus sp.
  promoter aprE ( CP011101 )
gtctattttcgttcttttcatgaaaatagttatttcgaagcgagagatgatatacctaaatagagataaaatcatctcaaaaaaatgggtctactaaaatattattccatctattacaataaattaacagaatagtcttttaaaagtctactctgaatttttaaaaggagagggtaaa

SEQ ID NO: 52 - DNA, 179 bp, Bacillus sp.
  promoter aprE ( CP002906 )
ctattttcgttcttttcatgaaaattgttatttcgaaacgagagatgatatacctaaatagagttaaaatcatcgcgaaaaaatgggtctactaaaatattaatccatctattacaataaatttacagaatagtccatctagggtctaatctgaatattttttaaaaggagagggtaaa

SEQ ID NO: 53 - DNA, 179 bp, Bacillus sp.
  promoter aprE ( CP003695 )
tctattttcgttcttttcatgaaaatagttatttcgaagcgagagatgatatacctaaatagagataaaatcatctcaaaaaaatgggtctactaaaatattattccatctattacaataaattcacagaatagtcttttaaaagtctactctgaatttttttaaaaggagagggtaaa

SEQ ID NO: 54 - DNA, 179 bp, Bacillus sp.
  promoter aprE ( CP004405 )
gtctattttcgttcttttcacgaaagtagttatttcgaagcgagagatgatatacctaaatagagataaaatcatctcaaaaaaatgggtctactaaaatattattccatctattacaataaattcacagaatagtcttttaaaagtctactctgaattttttaaaaggagagggtaaa

SEQ ID NO: 55 - DNA, 179 bp, Bacillus sp.
  promoter aprE ( Y14083 )
tctattttcgttcttttcatggaaatagttatttcgaagcgagagatgatatacctaaatagagataaaatcatctcaaaaaaatgggtctactaaaatattattccatctattacaataaattcacagaatagtcttttaaaagtctactctgaatttttttaaaaggagagggtaaa

SEQ ID NO: 56 - DNA, 179 bp, Bacillus sp.
  promoter aprE ( ADGS01000027 )
gtctattttcgttcttttcataaaattagttatttcgaagcgagagatgatatacctaaatagagataaaatcatcgcaaaaaaatgggtctactaaaatattattccatctattacaataaattaacagaatagtcttttaaaagtctactctgaattttttaaaaggagagggtaaa

SEQ ID NO: 57 - DNA, 194 bp, Bacillus sp.
  promoter aprE ( CP012600 )
aactctgcagatagatcgctataatgttaaataatgggttttaaaagaaactattttactaagttttcttcattggtatgcctacagaaaatgggtctactcgctttttctttcctgttttacaatatgtttacagaataatttgaaagcaggcgtgcccgctcgaaggttgttctgaaatgagaggagtgtga

SEQ ID NO: 58 - DNA, 173 bp, Bacillus sp.
  promoter aprE ( CP002905 )
cgttcttttctgtataaaattagttatttcgaagcgagagatgatatacctaaatagagataaaatcatcgcgaaaaaatgggtctactaaaatattattccatctattacaataaatttacagaatagtcttttaaaagtctactctgaattcttttaaaggagagggtaaa

SEQ ID NO: 59 - DNA, 173 bp, Bacillus sp.; identical to SEQ ID NO: 58
  promoter aprE ( CP018173 )

SEQ ID NO: 60 - DNA, 184 bp, Bacillus sp.
  promoter aprE ( CP013950 )
tccttccatcggttttttccattaaaatttaaatatttcggaaagagagttgatatacctaaatagaaataaaacgggatgaaaaaaattgggcctactaaaatattattccatactatacaattaatccacagaatagtcagtctattggttattctgcaaatgaaaaaaaggagaggataaa

SEQ ID NO: 61 - DNA, 184 bp, Bacillus sp.
  promoter aprE ( CP009748 )
tccatccatcggttttttccattaaaatttaaatatttcgaaaagagagatgatatacctaaatagaaataaaacaatctgaaaaaaattgggtctactaaaatattattccatactatacaattaatcaacagaataatctgtctattggttattctgcaaatgaaaaaaaggagaggataaa

SEQ ID NO: 62 - DNA, 184 bp, Bacillus sp.
  promoter aprE ( FN597644 )
tccatccatcggttttttccattaaaatttaaatatttcgaaaagagagatgatatacctaaatagaaataaaacaatctgaaaaaaattgggtctactaaaatattattccatactatacaattaatacacagaataatctgtctattggttattctgcaaatgaaaaaaaggagaggataaa

SEQ ID NO: 63 - DNA, 184 bp, Bacillus sp.
  promoter aprE ( CP002634 )
tccatccatcggttttttccattaaaatttaaatatttcgaaaagagagatgatatacctaaatagaaataaaacaatctgaaaaaaattgggtctactaaaatattattccatactatacaattaatccacagaataatctgtctattggttattctgcaaatgaaaaaaaggagaggataaa

SEQ ID NO: 64 - DNA, 184 bp, Bacillus sp.; identical to SEQ ID NO: 63
  promoter aprE ( CP016913 )

SEQ ID NO: 65 - DNA, 184 bp, Bacillus sp.
  promoter aprE ( ARYD01000002 )
tccttccatcggttttttccattaaaatttaaatatttcggaaagagagatgatatacctaaatagaaataaaacaaactgaaaaaaattgggtctactaaaatattattccatgctatacaattaatccacagaataatctgtctattggttgttctgcaaatgaaaaaaaggagaggataaa

SEQ ID NO: 66 - DNA, 159 bp, Bacillus sp.
  promoter aprE ( CP009938 )
aatttaaatatttcggaaagagagatgatatacctaaatagaaataaaacaaactgaaaaaaattgggtctactaaaatattattccatgctatacaattaatccacagaataatctgtctattggttgttctgcaaatgaaaaaaaggagaggataaa

SEQ ID NO: 67 - DNA, 159 bp, Bacillus sp.; identical to SEQ ID NO: 66
  promoter aprE ( CP006960 )

SEQ ID NO: 68 - DNA, 158 bp, Bacillus sp.
  promoter aprE ( CP014990 )
aatttaaatatttcggaaagagagatgatatacctaaatagaaataaaacaaactgaaaaaaattgggtctactaaaatattattccatactatacaattaatccacagaataatctgtctattggttattctgcaaatgaaaaaaggagtggataaa

SEQ ID NO: 69 - DNA, 184 bp, Bacillus sp.
  promoter aprE ( AJVF01000040 )
tccttccatcggttttttccattaaaatttaaatatttcggaaagagagttgatatacctaaatagaaataaaacgggatgaaaaaaattgggtctactaaaatattattccatactatacaattaatccacagaatagtctgtctattggttattctgcaaatgaaaaaaaggagaggataaa

SEQ ID NO: 70 - DNA, 158 bp, Bacillus sp.; identical to SEQ ID NO: 68
  promoter aprE ( AMPK01000010 )

SEQ ID NO: 71 - DNA, 184 bp, Bacillus sp.
  promoter aprE ( LN999829 )
gtccttccatcggttttttccattaaaatttaaatatttcggaaagagagatgatatacctaaatagaaataaaacaaactgaaaaaaattgggtctactaaaatattattccatactatacaattaatccacagaataatctgtctattggttattctgcaaatgaaaaaaggagtggataaa

SEQ ID NO: 72 - DNA, 184 bp, Bacillus sp.; identical to SEQ ID NO: 65
  promoter aprE ( AMQI01000005 )

SEQ ID NO: 73 - DNA, 159 bp, Bacillus sp.; identical to SEQ ID NO: 66
  promoter aprE ( CP017775 )

SEQ ID NO: 74 - DNA, 158 bp, Bacillus sp.; identical to SEQ ID NO: 68
  promoter aprE ( CP006952 )

SEQ ID NO: 75 - DNA, 184 bp, Bacillus sp.; identical to SEQ ID NO: 71
  promoter aprE ( CP007165 )

SEQ ID NO: 76 - DNA, 184 bp, Bacillus sp.; identical to SEQ ID NO: 71
  promoter aprE ( ANAT01000020 )

SEQ ID NO: 77 - DNA, 158 bp, Bacillus sp.; identical to SEQ ID NO: 68
  promoter aprE ( CP019040 )

SEQ ID NO: 78 - DNA, 184 bp, Bacillus sp.; identical to SEQ ID NO: 71
  promoter aprE ( CP017747 )

SEQ ID NO: 79 - DNA, 158 bp, Bacillus sp.; identical to SEQ ID NO: 68
  promoter aprE ( CP015911 )

SEQ ID NO: 80 - DNA, 184 bp, Bacillus sp.; identical to SEQ ID NO: 65
  promoter aprE ( CP009679 )

SEQ ID NO: 81 - DNA, 151 bp, Bacillus sp.
  promoter aprE ( HG514499 )
atatttcggaaagagagatgatatacctaaatagaaataaaacaaactgaaaaaattgggtctactaaaatattattccatgctatacaattaatccacagaataatctgtctattggttgttctgcaaatgaaaaaaaggagaggataaa

SEQ ID NO: 82 - DNA, 158 bp, Bacillus sp.
  promoter aprE ( CP011347 )
aatttaaatatttcggaaagagagatgatatacctaaatagaaataaaacaaactgaaaaaaattgggtctactaaaatattattccatgctatacaattaatccacagaataatctgtctattggttgttctgcaaaagaaaaaaggagtggataaa

SEQ ID NO: 83 - DNA, 184 bp, Bacillus sp.
  promoter aprE ( CP006890 )
gtccttccatcggttttttccattaaaatttaaatatttcggaaagagagatgatatacctaaatagaaataaaacaaactgaaaaaaattgggtctactaaaatattattccatgctatacaattaatccacagaataatctgtctattggttgttctgcaaaagaaaaaaggagtggataaa

SEQ ID NO: 84 - DNA, 158 bp, Bacillus sp.
  promoter aprE ( AJST01000001 )
aatttaaatatttcggaaagagagatgatatacctaaatagaaataaaacaaactgaaaaaaattgggtctactaaaatattattccatgctatacaattaatccacagaataatctgtctattggttgttctgcaaatgaaaaaaggagtggataaa

SEQ ID NO: 85 - DNA, 159 bp, Bacillus sp.
  promoter aprE ( AFSU01000055 )
aatttaaatatttcggaaagagagatgatatacctaaatagaaataaaacaaattgaaaaaaattgggtctactaaaatattattccatgctatacaattaatccacagaataatctgtctattggttgttctgcaaatgaaaaaaaggagaggataaa

SEQ ID NO: 86 - DNA, 159 bp, Bacillus sp.
  promoter aprE ( CP015417 )
aatttaaatatttcggaaagagagatgatatacctaaatagaaataaaacaaattgaaaaaaattgggtctactaaaatattattccatgctatacaattaatccacagaataatctgtttattggttgttctgcaaatgaaaaaaaggagaggataaa

SEQ ID NO: 87 - DNA, 184 bp, Bacillus sp.
  promoter aprE ( HG328254 )
tccttccatcggttttttccattaaaatttaaatatttcggaaagagagatgatatacctaaatagaaataaaacaaattgaaaaaaattgggtctactaaaatattattccatgctatacaattaatccacagaataatctgtttattggttgttctgcaaatgaaaaaaaggagaggataaa

SEQ ID NO: 88 - DNA, 184 bp, Bacillus sp.
  promoter aprE ( HF563562 )
tccttccatcggttttttccattaaaatttaaatatttcggaaagagagatgatatacctaaatagaaataaaacaaattgaaaaaaattgggtctactaaaatattattccatgctatacaattaatccacagaataatctgtctattggttgttctgcaaatgaaaaaaaggagaggataaa

SEQ ID NO: 89 - DNA, 152 bp, Bacillus sp.
  promoter aprE ( CP006845 )
atatttcggaaagagagatgatatacctaaatagaaataaaacaaactgaaaaaaattgggtctactaaaatattattccatgctatacaattaatccacagaataatctgtctattggttgttctgcaaatgaaaaaaaggagaggataaa

SEQ ID NO: 90 - DNA, 184 bp, Bacillus sp.; identical to SEQ ID NO: 65
  promoter aprE ( CP000560 )

SEQ ID NO: 91 - DNA, 159 bp, Bacillus sp.
  promoter aprE ( CP014783 )
aatttaaatatttcggaaagagagatgatatacctaaatagaaataaaacaaattgaaaaaaattgggtctactaaaatattattccatgctatacaattaatctacagaataatctgtctattggttgttctgcaaatgaaaaaaaggagaggataaa

SEQ ID NO: 92 - DNA, 192 bp, Bacillus sp.
  promoter aprE ( CP011007 )
aatagtagaataaacatattttaccatcatttccaagtgacttaattccccaattttcgctaggactttcacaaaaattcaggtctactcttatttgcctacttccattaaactgaatatacagaatagtcaaacggatcattcttatagactacggatgattattctgaaataaaaaaaagggatgtggat

SEQ ID NO: 93 - DNA, 159 bp, Bacillus sp.; identical to SEQ ID NO: 85
  promoter aprE ( CP007242 )

SEQ ID NO: 94 - DNA, 192 bp, Bacillus sp.
  promoter aprE ( APAS01000011 )
aaatacagaataaacatattttaccataatttccaagcgacttaattccctatttttcgctaggacttccacaaaaattcgggtctactcttatttgcctatctctattaaactgaaaatacagaataatcaaacggatcattctaatagactacggatgattattctgaaataagaaaaagggatgtggaa

SEQ ID NO: 95 - DNA, 192 bp, Bacillus sp.
  promoter aprE ( CP009108 )
aaatacacaataaacatattttaccataatttccaagcgacttaattccctatttttcgctaggacttccacaaaaattcaggtctactcttatttgcctacctccattaaactgaaaatacagaataatcaaacggatcattcttatagactacggatgattattctgaaataagaaaaagggatgtggaa

SEQ ID NO: 96 - DNA, 192 bp, Bacillus sp.
  promoter aprE ( CP012482 )
aaatacagaataaacatattttaccatcatttccaagcgacttaattccctatttttcgctaggacttccacaaaaattcgggtctactcttatttgcctatctctattaaactgaaaatacagaataatcaaacggatcattctaatagactacggatgattattctgaaataagaaaaagggatgtggaa

SEQ ID NO: 97 - DNA, 192 bp, Bacillus sp.
  promoter aprE ( CP000813 )
aacagtagaataaacatattttaccactatttccaagtgacttaattccccaattttcgctaggactttcacaaaaattcaggtctactcttatttgcctacttcccttaaactgaatatacagaataatcaaacgtctcattcttatagactacggatgattattctgaaataaaaaaaagggatgtggat

SEQ ID NO: 98 - DNA, 192 bp, Bacillus sp.
  promoter aprE ( AUYP01000015 )
aatgatagaataaacatattttaccaccagttccaagtgacttaattccccaattttcgctaggactttcacaaaaattcgggtctactcttatttgcctacttcccttaaactgaatatacagaataatcaaacggatcattcttatagactacggatgattattctgaaataagaaaaagggatgtggaa

SEQ ID NO: 99 - DNA, 192 bp, Bacillus sp.
  promoter aprE ( CP010997 )
aaatatagaataaacatattttaccatcatttccaagcgacttaattccctatttttcgctaggacttccacaaaaattcgggcctactcttatttgcctatctctattaaactgaaaatacagaataatcaaacggatcattctaatagactacggatgattattctgaaataagaaaaagggatgtggaa

SEQ ID NO: 100 - DNA, 192 bp, Bacillus sp.
  promoter aprE ( AMDH01000008 )
aaatataaaataaacatattttaccatcaattccaagcgacttaattccctatttttcgctaggacttccacaaaaattcaggtctactcttatttgcctatctctattaaactgaaaatacagaataatcaaacggatcattctaatagactacggatgattattctgaaataagaaaaagggatgtggaa

SEQ ID NO: 101 - DNA, 192 bp, Bacillus sp.
  promoter aprE ( AGBY01000040 )
aaatataaaataaacatattttaccatcatttccaagtgacttaattccctatttttcgctaggacttccacaaaaattcaggtctactcttatttgcctatctctattaaactgaaaatacagaataatcaaacggatcattctaatagactacggatgattattctgaaataagaaaaagggatgtggaa

SEQ ID NO: 102 - DNA, 192 bp, Bacillus sp.
  promoter aprE ( AP014928 )
aaatacagaataaacatattttaccatcatttccaagcgacttaattccctatttttcgctaggacttccacaaaaattcaggtctactcttatttgcctatctctattaaactgaaaatacagaataatcaaacggatcattctaatagactacggatgattattctgaaataagaaaaagggatgtggaa

SEQ ID NO: 103 - DNA, 192 bp, Bacillus sp.
  promoter aprE ( CP007436 )
aaatataaaataaacatattttaccatcatttccaagcgacttaattccctatttttcgctaggacttccacaaaaattcaggtctactcttatttgcctatctctattaaactgaaaatacagaataatcaaacggatcattctaatagactacggatgattattctgaaataagaaaaagggatgtggaa

SEQ ID NO: 104 - DNA, 192 bp, Bacillus sp.
  promoter aprE ( CP016784 )
aacagtagaacaaacatatttccccaacgtttccaagtgacttaattccccaattttcgctaggactttcacaaaaattcgggtctactcttatttgcctacttcccttaaactgaatatacagaataatcaaacgaatcattcttatagactacgaatgattattctgaaataagaaaaagggatgtggat

SEQ ID NO: 105 - DNA, 192 bp, Bacillus sp.
  promoter aprE ( ABRX01000001 )
aatagtagtataaacatattttaccatcatttccaagtgacttaattccccaattttcgctaggactttcacaaaaattcgggtctactcttatttgcctacttcccttaaactgaatatacagaatagtcaaacggatcattcttatagactacggatgattattctgaaataaaaaaaagggatgtggat

SEQ ID NO: 106 - DNA, 192 bp, Bacillus sp.
  promoter aprE ( AMSH01000033 )
aaatatagaataaacataatttaccataatttccaagcgacttaattccctatttttcgctaggactttcacaaaaattcaggcctactcttatttgcctatctcccttaaactgaaaatacagaataatcaaacgggtcattcttatagactatagatgattattctgaaataagaaaaagggatgtggaa

SEQ ID NO: 107 - DNA, 192 bp, Bacillus sp.
  promoter aprE ( CP018197 )
aatgatagaataaacatattttaccaccagttccaagtgacttaattccccaattttcgctaggactttcacaaaaattcgggtctactcttatttgcctacttcccttaaactgaatatacagaataattaaacggatcattcttatagactacggatgattattctgaaataagaaaaagggatgtggaa

SEQ ID NO: 108 - DNA, 192 bp, Bacillus sp.; identical to SEQ ID NO: 98
  promoter aprE ( AUPF01000014 )

SEQ ID NO: 109 - DNA, 192 bp, Bacillus sp.
  promoter aprE ( CP015607 )
aatgatagaataaacatattttaccaccggttccaagtgacttaattccccaattttcgctaggactttcacaaaaattcgggtctactcttatttgcctacttcccttaaactgaatatacagaataatcaaacggttcattcttatagactacggatgattattctgaaataaaaaaaagggatgtggaa

SEQ ID NO: 110 - DNA, 192 bp, Bacillus sp.
  promoter aprE ( CP010075 )
aatgatagaataaacatattttaccaccagttccaagtgacttaattccccaattttcgctaggactttcacaaaaattcaggtctactcttatttgcctacttcccttaaactgaatatacagaatagtcaaacggatcattctattagactacggatgattattctgaaataagaaaaagggatgtggaa

SEQ ID NO: 111 - DNA, 192 bp, Bacillus sp.
  promoter aprE ( CP015610 )
aatgatagaataaacataatttaccatcaattccaagtgacttaattccccaattttcgctaggactttcacaaaaattcaggtctactcttatttgcctacttcccttaaactgaatatacagaataatcaaacggatcattcttatagactacggatgattattctgaaataaaaaaaagggatgtggaa

SEQ ID NO: 112 - DNA, 198 bp, Bacillus sp.
  promoter aprE ( ACPC01000009 )
tcattccacctacaatttacaggggaaacaaggaagttatgacagatattcctattgggaaaataactctcgtttttctcctttaagagcgacttctgcctagtgttttcccaatatgaaaactagctgtcaggtgataaactgtccttaatagttctcaatagtgagaattattaaaactttattcaaggagtgagt

SEQ ID NO: 113 - DNA, 179 bp, Bacillus sp.
  promoter aprE ( ACPC01000009 )
aaaactaaaaaatactgccactaaccttggcgatgcgttctattatggacatggtgtaattaacgttgaaagtgctttgcaataactaagttgtcccccctttgaatagaagcaccaggatcaactcttaaatagagaagacactggtgctttataaattttttataggagagtgagct

SEQ ID NO: 114 - DNA, 196 bp, Bacillus sp.
  promoter aprE ( AULJ01000079 )
aatgcttgttctactatgatttttcgaggggatttgactcaaaccgcaaaatcccttccttatgtagtacttctgtcccttttaagaaaagaatgcaaaccaaaacactgttatgcctcttatattaaggatgatgttgcaaagatttctccagaaaaatattgtacgattattctgaaaacaatattaggaggga

SEQ ID NO: 115 - DNA, 171 bp, Bacillus sp.
  promoter aprE ( U39230 )
aatgacttatttttgaagttattaccatgatgcaccattgggaaattcgggtgtagaatctccggattaggccgccatatccagaccgctttcccaataacaattcctatattgttatgctaagttaactaaataatttgattatttagaaaaaaagaaggagcgtgaatt

SEQ ID NO: 116 - DNA, 107 bp, Bacillus sp.
  promoter aprE ( CP015610 )
caactagatcaaataaatggaaaaatttaaaaaataggacaaacggtcgtttctttttgtctaaaaatgtagttaaatgacaaaaagaaacgaaagggagagtgggt

SEQ ID NO: 117 - DNA, 176 bp, Bacillus sp.
  promoter aprE ( ANNK01000144 )
tttaaaattaatgaatagataaatatgtttatcgaagtatatagcctactttaaaacattgaggatgtttgtgtcttgcaaaaagatttcctttttaataatacgtcttttaagcggtcctgctagatttgctgtaagggcttatctaatcttactcaaagaaagaggaggttcga

SEQ ID NO: 118 - DNA, 108 bp, Bacillus sp.
  promoter aprE ( CP010075 )
caactagatcaaataaatggaaaaatttaaaaaataggacaaacggtcgtttctttttgtctaaaaatgtagttaaatgacaaaaagaaacgaaagggagagtgagta

SEQ ID NO: 119 - DNA, 158 bp, Bacillus sp.
  promoter aprE ( AUPF01000001 )
taatgcttatgatcactatcaagaggatcataagcatttttattttgcgacaactagatcaaataaatggaaaaatttaaaaaataggacaaacggtcgtttctttttgtctaaaaatgtagttaaatgacaaaaagaaacgaaagggagagtgagta

SEQ ID NO: 120 - DNA, 108 bp, Bacillus sp.
  promoter aprE ( CP015607 )
caactagatcaaataaatggaaaaatttaaaaaataggacaaacggtcgtttctttttgtctaaaaatgtagttaaatgacaaaaagaaacgaaagacgaaagggaga

SEQ ID NO: 121 - DNA, 108 bp, Bacillus sp.; identical to SEQ ID NO: 118
  promoter aprE ( CP018197 )

SEQ ID NO: 122 - DNA, 158 bp, Bacillus sp.
  promoter aprE ( AUYP01000069 )
aaatgcttatgatcactatcaagaggatcataagcatttttattttgcgacaactagatcaaataaatggaaaaatttaaaaaataggacaaacggtcgattctttttgtctaaaaatgtagttaaatgacaaaaagaaacgaaagggagagtgagta

SEQ ID NO: 123 - DNA, 157 bp, Bacillus sp.
  promoter aprE ( AMDH01000012 )
gaatgcttatgatcgctatgaacaggatcataagcatttttattttgcgacaagtagatcaaataattggaaaaatttaaaaaataggacaaacggtcgtttctttttgtctaaaaatgtagttaaatgacaaaaagaaatgaaagggagagtgggt

SEQ ID NO: 124 - DNA, 157 bp, Bacillus sp.; identical to SEQ ID NO: 123
  promoter aprE ( AGBY01000013 )

SEQ ID NO: 125 - DNA, 157 bp, Bacillus sp.; identical to SEQ ID NO: 123
  promoter aprE ( AMBY01000003 )

SEQ ID NO: 126 - DNA, 107 bp, Bacillus sp.
  promoter aprE ( CP009108 )
caagtagatcaaataattggaaaaatttaaaaaataggacaaacggtcgtttctttttgtctaaaaatgtagttaaatgacaaaaagaaatgaaagggagagtgggt

SEQ ID NO: 127 - DNA, 157 bp, Bacillus sp.; identical to SEQ ID NO: 123
  promoter aprE ( APAS01000018 )

SEQ ID NO: 128 - DNA, 106 bp, Bacillus sp.
  promoter aprE ( ABRX01000005 )
caaccaattcaaataaatggaaaaatttaaaaaataggacaaacggtcgtttctttttgtctaaaaatgtagttaaatgacaaaaagatgaaagggagagtgagta

SEQ ID NO: 129 - DNA, 108 bp, Bacillus sp.
  promoter aprE ( AMSH01000086 )
caagtagatcaaataaatggaaaattttaaaaaataggacaaatggtcgtttctttttgtctaaaaatgtagttaaatggcaaaaagcaatgaaagggagagtgagta

SEQ ID NO: 130 - DNA, 107 bp, Bacillus sp.; identical to SEQ ID NO: 126
  promoter aprE ( CP012482 )

SEQ ID NO: 131 - DNA, 107 bp, Bacillus sp.
  promoter aprE ( AP014928 )
caagtagatcaagtaattggaaaaatttaaaaaataggacaaacggtcgtttctttttgtctaaaaatgtagttaaatgacaaaaagaaatgaaagggagagtgggt

SEQ ID NO: 132 - DNA, 108 bp, Bacillus sp.; identical to SEQ ID NO: 129
  promoter aprE ( CP017786 )

SEQ ID NO: 133 - DNA, 191 bp, Bacillus sp.
  promoter aprE ( CP001878 )
catataaaactatttatattactatatttattaaatgtggaataatagacgagaatcctcctattattctagtataataacttgtcacagaaaatacatacttttttgaattttgatagattattcataggaggtgatcgcctatgaatcttcaaaaaatacgctcagcgttgaaggttaagcaatcggca

SEQ ID NO: 134 - DNA, 103 bp, Bacillus sp.
  promoter aprE ( CP011007 )
caaccagttcaaatggaaaaatttttaaaaataggacaaacgatcgtttctttttgtctgaaaatgtagttaaatgacaaaaagatgaaagggagagtgagta

SEQ ID NO: 135 - DNA, 106 bp, Bacillus sp.
  promoter aprE ( CP016784 )
caactagttcaaataaatggaaaaatttaaaaaataggacaaacggtcgtttctttttgtctgaaaatgtagttaaatgacaaaaagatgaaagggagagtgagta

SEQ ID NO: 136 - DNA, 170 bp, Bacillus sp.
  promoter aprE ( AUCJ01000007 )
agacctcgaccttttgtcatgaaggggacggatgtctcgaatcatgttgaaaaaaatgtaaataaaacagtaagttatctgtaaatttgaaatttttgttgaattatcctccttttccttgctaaagtacgagttacaactttttctaattcacaagtgggaagggagaa

SEQ ID NO: 137 - DNA, 170 bp, Bacillus sp.
  promoter aprE ( ARIV01000026 )
gggatcgtaaaaagaaatcaaatgtttcgtgtgacgaagaattaatatctacaattaagagtaaataagtaattttatgggattgcgataaaaaaatatccgaatatcgaaaattcggatgttcgaatttgttgctacgctatatgtaatctttactaggaggaatgtcg

SEQ ID NO: 138 - DNA, 189 bp, Bacillus sp.
  promoter aprE ( ATVM01000028 )
aactatatttatagataattataatagtaatagttcttatcattaaataatagaaaatccaaagaaagacctgagtccattctcgaaatcaatagctctttcttcttcaatgtctctcttttggttgaaatgtaaaaattatccttcaaactagaacaaatacattttagggggaagaacatgagaacg

SEQ ID NO: 139 - DNA, 179 bp, Bacillus sp.
  promoter aprE ( CP001034 )
ggtaattttcagactattcacaaatttatgcaggtttttgccagaagacaaacacaaaatcataaataaattctttttttagaaaggagggattacaaaatattttaaaaccagtccccaagtaagtgtagataattgttaggtgagtgtagttaaattgtttaagtgtagctttgaaa

SEQ ID NO: 140 - DNA, 193 bp, Bacillus sp.
  promoter aprE ( BA000004 )
aaagggaggagtggtgcaattttcaagcaattgattgacatatataaatatttttatataggtccgctgttgaaaaagaataacgaagcatgaaagatactaggtcagttgctgtggaaatatggcggatcagagaatgtgagatctgtgatagattaagaacaacttctagtagagggaggaaggtagaatg

SEQ ID NO: 141 - DNA, 186 bp, Bacillus sp.
  promoter aprE ( AKIF01000009 )
aacataaccgatccactatattccttgctttacagagtaaacaacgcttaaaggaggttttaagggaggtttataccattcaacaatatctcaacctgattcatttatgctgaggtttgcttcacctaactagcacgttaatacccacacatatttataaatcttttatttaggagtgtttaaatg

SEQ ID NO: 142 - DNA, 164 bp, Bacillus sp.
  promoter aprE ( AUCJ01000007 )
catgggaaagttggcggggtctttggtttaatacttttatctaccaagaaaataggtccaatcatggtaataaatagaatctatcatactatatttagtggtattattttaattgttcagaatagtctatctattttagatcaaaaaagagtagggagtggttg

SEQ ID NO: 143 - DNA, 189 bp, Bacillus sp.
  promoter aprE ( CP003923 )
aacgatttattaataactttcccaatataaaaagacttatatgacggcttatttagttcattcttcttaattatcctttcctccttattcaataaatacctttttatcgattcatgtttcccaataaaaaggaggttcattgcgtgctatcgtaatggacaacccattttgaataggaggcgttaccga

SEQ ID NO: 144 - DNA, 166 bp, Bacillus sp.
  promoter aprE ( CP016020 )
gatgggctacagttggaaacccatattatatcagtaactaagaacaaaaaaggatagactgagggatttttataatctactcacctatccttttttactaatagtttagaattttaggagaatagaaatgggtatatacttaaaaattctgaagggacaattgtat

SEQ ID NO: 145 - DNA, 176 bp, Bacillus sp.
  promoter aprE ( AUCJ01000005 )
ttactagtttaccagatctttgaaaggagggttgttttatgttgaaaaaactcgttgcgattgctcttgttgtcggttttcgcaaatatcgtctctgttcaagcttcttttgacccagcgccacaggatttgccttacgaaagctaatttcattttataaaaaaggagagggttgt

SEQ ID NO: 146 - DNA, 189 bp, Bacillus sp.
  promoter aprE ( CP011366 )
gaatttttaaatatacttaaatactaggatttaaatccatgctagcatacatggggaaaagaatgacaatattcggctctatcagaatcttattcccctcattcataaaaatggacaaactttttgcataactggatataacatatttatcatgtttattatggagatagactgaaagaagggggaaat

SEQ ID NO: 147 - DNA, 192 bp, Bacillus sp.
  promoter aprE ( ANAM01000008 )
ccttactaattaaaacatatataaaaactaggaattataatgatgatagcataccgatggtcagggaaccaaataggaagcttgcacagttgaattgtttcccctgtgtgaaaacggacaaagtttttgcacacctgaatatagcacatttataagatttatcatggagatagattagacaaagggggaaac

SEQ ID NO: 148 - DNA, 192 bp, Bacillus sp.
  promoter aprE ( AUHI01000001 )
ggaaactaaatcaaaagtcgggtttgaaattatttttaattatcatataattcaaatcattccaaaaaataagtcgggtggaacatacatattgtttagattcgtaatgctcccccccttttaattgtataacgatctatatgatttgcttcctacctgacttattttaaattagaaaaggagtggagacat

SEQ ID NO: 149 - DNA, 174 bp, Bacillus sp.
  promoter aprE ( BA000004 )
taagaaaaaaaacgatatataaatatgtttatctaagtgggaagttttgtagaataatgacggttattgaccataggaagtggacaaaccaccaaaaaatacagactcctttgcaatctgctagattgaataataagagatttctgaaatatctgctagatcgaaaagttctga

SEQ ID NO: 150 - DNA, 188 bp, Bacillus sp.
  promoter aprE ( ALXI01000002 )
agggactaagttcagttgaatatgtctgccagaagccgaggatattaggattaaattttaaaagctattctccggaatagcttttttatataaatatgtacttaattatgggggaatcagaaagaaaagttaagaaaagattaatatctagagatcttcataaagaaatattaaggggggaatacaaa

SEQ ID NO: 151 - DNA, 200 bp, Bacillus sp.
  promoter aprE ( CP001615 )
tttgttcatctaatatttcaattcatttttcgtacaattgagtcaaaagtcttgtatctgaatcccctgtatccttatctaaatattcagatatttaatgccattggcgtattctacacgttggcaaaacttgttactctatggtcacggcagttggacatcccgtcatcttgcctacataccttaaaggaggaaacaag

SEQ ID NO: 152 - DNA, 145 bp, Bacillus sp.
  promoter aprE ( ATKK01000024 )
gatatccccgattccgcattttaaatattctgaatattgatacgattgacatctacatcccgatggaaatacttgttacattatggacacggcaaacaaacgtcacttcatcacgcctacataacctaaagggggaaccacattg

SEQ ID NO: 153 - DNA, 163 bp, Bacillus sp.
  promoter aprE ( CP007453 )
tatgtaaatcctggggtctatcagggagcggtcagacattataattattaagataacatctgatgaaggaagaaaccttcttcaaatgaaacttataaactatatagctcttagtggctatatcattattatactaggtggtattattatgaaattcaaaaaa

SEQ ID NO: 154 - DNA, 200 bp, Bacillus sp.
  promoter aprE ( ATCL01000009 )
tttatcggattcgatttggcgttctgctgccatctccttgagtcaaaagtcttgtatctgaatcccctgtatccttatctaaatattcagatttttaattccattgacatattttacccgatgggaaaacttgttactctatgggcacggcagttcgacatcgcgtcacctcgcctacataacccgaaggaggaaacaag

SEQ ID NO: 155 - DNA, 189 bp, Bacillus sp.
  promoter aprE ( CP006777 )
ctgtcactattcgtttgaatagttttttgttatatggaattataataaaattaatcaaatctgtttatcagtacagttccttagaagtgcagataaaatttataggaggagggaataaggtctaaaagagtggtgataagcttaattaagcacatttacgaaagataaatttaaggaggggtatataaa

SEQ ID NO: 156 - DNA, 66 bp, Bacillus sp.
  promoter aprE ( CP001638 )
aaatagtaaactaagagcgtaatcacaaaagcagttaaacaagcggcagattttttgcatgagagg

SEQ ID NO: 157 - DNA, 199 bp, Bacillus sp.
  promoter aprE ( AGBD01001966 )
gacaatagagaattacttagcttcacgaacttgaagggtggggaccagcctaaacgattcgaagtcgatttatctggggtttccacgttgcaaatcttgtatacaagggacaccggatcggatcttacgattgatattgccgatgctattttacatcacaaataaccagcctggaggaacatagaatgaaaataatccg

SEQ ID NO: 158 - DNA, 199 bp, Bacillus sp.
  promoter aprE ( AJTY01000058 )
gacaatagagaattacttagcttcatgaacttgaagggtggggaccagcctaaacgattcgaagtcgatttatctggggtttccacgttgcaaattttgtatacaaaggacaccggctcggatcttacgattgatattgccgatgctattttacatcacaaataaccagcctggaggaacatagaatgaaaataatccg

SEQ ID NO: 159 - DNA, 172 bp, Bacillus sp.
  promoter aprE ( AMRO01000052 )
tgccaaaaagttaggctcttcatacaaagctatttatctcggaatattagattacgcatccgaatcaggattgaactttcagttatacgaccgtgttcaatgaaatattataccgccgtgttcaatgaaatagtaaaatagagaatagtagcagattttctcgcatgagagg

SEQ ID NO: 160 - DNA, 163 bp, Bacillus sp.
  promoter aprE ( CP009502 )
ctggcgaaattcaataaacttttgagatttatgatttacagaaataggtacactgccttagctacaggaatacagagaacctgcgttaatatattcaatcatattatgtttattgtgtaatataatttagcaaatattatataaaataaagaatactagtata

SEQ ID NO: 161 - DNA, 137 bp, Bacillus sp.
  promoter aprE ( CP009517 )
aagtcaagtttttgtacgttgtggataattttgaccttttgatatatgtttgcaataaatactcaaataatgctaattatgtttattttgtcataatatttggcaagtgttatataaaataatgaatatttgaatat

SEQ ID NO: 162 - DNA, 163 bp, Bacillus sp.
  promoter aprE ( CP009501 )
ctggcgagattcaataaacttttgagatttatgatttacagaaataggtacactgccttagctacaggaatacagagaacctgcgttaatatattcaatcatattatgtttattgtgtaatataatttagcaaatattatataaaataaagaatactagtata

SEQ ID NO: 163 - DNA, 179 bp, Bacillus sp.
  promoter aprE ( AMSQ01000022 )
tttattttataaccatttttaaaaattaattatttttgtaaaatagtgatttttacacagaattaacaaaaacccctaaatataaagtttattataaaattaaatattcgtattgtttctttttctaaaataacctatattaatacatgtaccttaaaagatagaatagaaatggggtc

SEQ ID NO: 164 - DNA, 180 bp, Bacillus sp.
  promoter aprE ( AKGE01000032 )
tttattttataaccattttttaaaaattaattatttttgtaaaatagtgatttttacacagaattaacaaaaacccctaaatataaagtttattataaaattaaatattcgtattgtttctttttctaaaataacctatattaatacatgtaccttaaaagatagaatagaaatggggtc

SEQ ID NO: 165 - DNA, 196 bp, Bacillus sp.
  promoter aprE ( CP003362 )
gttcattaactaacctgcatggatattgtgctcctgcatccagatgacgggacgggcagctaagtagccagcatgccggatatataattgcagtaataacaaacagttaatttatttttcaatgaaaagtaaataaacctatatatatacgaccataatatattcaatgggtgttaccataggaaagcaaacaaaa

SEQ ID NO: 166 - DNA, 188 bp, Bacillus sp.
  promoter aprE ( ASZU01000003 )
attctgctaatttctgcctgctagcacgtcgagtctcctcgccgggggacagatcactcacaggcaccgcttgactcatgacaacaggtgtattgtgctgacgcaaaccatgttcttctgcatcggaaccacttacccaaccaatcaaatccacaaatcaaaaggaggatgttttcgtgaagaaacgc

(19) United States
(12) Patent Application Publication
(10) Pub. No.: US 2022/0186177 A1
(43) Pub. Date: Jun. 16, 2022

(54) INDUSTRIAL FERMENTATION PROCESS FOR BACILLUS USING DEFINED MEDIUM AND MAGNESIUM FEED

(71) Applicant: BASF SE, Ludwigshafen (DE)

(72) Inventors: Andreas Daub, Ludwigshafen (DE); Aydin Golabgir Anbarani, Ludwigshafen (DE); Tobias Klein, Ludwigshafen (DE); Stephan Freyer, Ludwigshafen (DE); Thomas Kaeding, Lampertheim (DE); Max Fabian Felle, Ludwigshafen (DE); Paul Igor Costea, Ludwigshafen (DE)

(21) Appl. No.: 17/432,204

(22) PCT Filed: Feb. 18, 2020
(86) PCT No.: PCT/EP2020/054172

§ 371 Date: Aug. 19, 2021

(30) Foreign Application Priority Data
Feb. 20, 2019 (EP) 19158372.3
Dec. 12, 2019 (EP) 19215651.1

(51) Int. Cl. C12N 1/38; C12P 21/02; C12N 1/20

(52) U.S. Cl. CPC C12N 1/38; C12N 9/54; C12N 1/205; C12P 21/02

(57) ABSTRACT

The present invention is directed to an industrial fermentation process for cultivating a Bacillus cell in a chemically defined fermentation medium and a method for producing a protein of inter-est comprising the steps of providing a chemically defined fermentation medium, inoculating the fermentation medium with a Bacillus cell comprising a gene encoding a protein of interest, cultivating the Bacillus cell in the fermentation medium under conditions conductive for the growth of the Bacillus cell and the expression of the protein of interest, wherein the cultivation of the Bacillus cell comprises the addition of one or more feed solutions comprising one or more chemically defined carbon sources and magnesium ions to the fermentation medium.

### FIELD OF THE INVENTION

The present invention is directed to an industrial fermentation process for cultivating a Bacillus cell in a chemically defined fermentation medium and a method for producing a protein of interest comprising the steps of providing a chemically defined fermentation medium, inoculating the fermentation medium with a Bacillus cell comprising a gene encoding a protein of interest, cultivating the Bacillus cell in the fermentation medium under conditions conductive for the growth of the Bacillus cell and the expression of the protein of interest, wherein the cultivation of the Bacillus cell comprises the addition of one or more feed solutions comprising one or more chemically defined carbon sources and one or more feed solutions containing magnesium ions to the fermentation broth.

### BACKGROUND OF THE INVENTION

Microorganisms of the Bacillus genus are widely applied as industrial workhorses for the production of valuable compounds, especially proteins like washing- and/or cleaning-active enzymes. The biotechnological production of these useful substances is conducted via fermentation and subsequent purification of the product. Bacillus species are capable of secreting significant amounts of protein to the fermentation broth. This allows a simple product purification process compared to intracellular production and explains the success of Bacillus in industrial application.

Industrial fermentation is typically performed in large fermenters (working volume greater than 1 m³) under aerobic conditions by controlling several process variables, including but not limited to aeration rate, stirring speed, pH, initial concentrations of various nutrients, and feeding rate profiles of one or more nutrients. To grow and produce products of interest, the microorganisms require several macronutrients, e.g., carbon, nitrogen, phosphor, sulfur, in addition to micro-nutrients, such as trace elements, e.g., iron, copper, manganese, zinc, etc., and vitamins. These nutrients can be provided in the fermentation medium or supplemented throughout the fermentation process via one or more feeding solutions.

Fermentation processes that are relevant for industrial application involve supplementation of large amounts of carbon source to the cells to ensure the availability of sufficient amounts of growth and availability of precursors for the product of interest. In most cases, the amount of supplied carbon source exceeds 200 g of the pure component (e.g., glucose) per initial volume of fermentation medium used in the fermentation process.

Generally, fermentation processes can be performed with either complex or chemically defined media. Complex media involve the utilization of complex raw materials, such as soybean meal, soybean hydrolysate, and corn steep liquor. The complex raw materials contain a mixture of proteins, carbohydrates, lipids, vitamins, minerals and other biologically relevant molecules. The complex raw materials are not chemically defined. On the other hand, a defined media process uses known amounts of chemically defined components as the source of nutrients for the microorganisms. Using complex media in fermentation processes can have advantages with respect to availability, and simultaneous provision of nutrients to the cells, such as trace elements and vitamins. This property of containing a diverse set of nutrients can be useful in cases the exact nutritional requirements of the microorganisms is unknown. However, using complex raw materials also has clear disadvantages. First, processes that use complex raw materials are prone to larger deviations in their outcomes (quality attributes), such as product titer and product purity, due to seasonal and geographic variation in the quality of the complex raw materials. Second, complex raw materials negatively influence downstream processing increasing processing costs. For example, solids content in the fermentation broth may be increased leading to higher effort in biomass separation. Complex raw materials also lead to color formation and influence the smell of the product which necessitates an increased effort for decolorisation and deodoration. Furthermore, using complex raw materials makes it more difficult to analyze important quality characteristics of the fermentation process. For instance, once complex raw materials with insoluble components are used, traditional approaches to measure the biomass content of the fermentation process become ineffective. Therefore, fermentation processes that use chemically defined media provide clear benefits with respect to improved consistency of quality and superior possibilities to characterize and analyze the process.

For these reasons the fermentation industry has moved away from complex raw material based production processes to chemically defined media production processes in the last decades whenever this was possible, i.e., when the nutritional requirements of the industrial microorganism could be met with a defined media process. US20140342396A1 gives examples for the production of various valuable products based on defined media processes with a wide range of organisms: glucose isomerase production with Streptomyces lividans, penicillin V production with Penicillium chrysogenum, 7-ADCA production with Penicillium chrysogenum, lovastatin production with Aspergillus terreus, clavulanic acid production with Streptomyces clavuligerus, amyloglucosidase production with Aspergillus niger, Astaxanthin production with Phaffia rhodozyma, arachidonic acid production with Mortierella alpina, erythromycin production with Saccharopolyspora erythraca, β-carotene production with Blakeslee trispora. However, a production process with a Bacillus species is not disclosed in US20140342396A1.

WO9110721A2 shows an example of using chemically defined media for the production of biomass for Escherichia coli. The process does not teach relevant information for devising a process for protein production with Bacillus.

Defined media have been used for Bacillus species for scientific purposes in small scale lab processes. These processes are characterized by scale of less than 50 liter, low biomass concentration and low concentration of carbon source, naturally resulting in low productivity. Hence, these processes are not relevant for industrial application and they do not provide any teaching on how to establish an industrially relevant process based on defined media. For instance, EP0406711A1 teaches the production of subtilisin with Bacillus licheniformis DSM 1969 with chemically defined medium with an ammonium limited process control strategy. Ammonium was controlled to a very low concentration of 0.15 mM (0.26 mg/L) by a closed-loop control necessitating continuous measurement of the ammonia concentration during the process. However, the approach is not relevant for an industrial production process because the amount of biomass and carbon source is lower (92 g carbon source per liter) than the amount needed for fermentation processes with Bacillus that can be considered industrially relevant. In addition, the proposed process with ammonia limitation is too complex to be easily transferred to a production environment. For instance, there is no reliable online probe for ammonia available that could be used under sterile conditions in production and manual sampling to reliably control the ammonia concentration to the low values needed for the proposed process is not desirable in routine production.

In EP0631585B1 an attempt was made to overcome the problems of using a minimal fermentation medium in industrial fermentation of Bacillus cells by adding ammonium sulfate in order to precipitate the protein of interest during the fermentation process. In EP0631585B1 it is stated that without the precipitation the use of a minimal medium is no alternative to complex medium. However, due to the precipitation of the protein of interest the process described in EP0631585B1 does not allow for an easy separation of the protein of interest from the biomass.

Thus, for industrially relevant production of proteins using Bacillus species to-date, it has been generally accepted that utilization of a chemically defined medium is not possible and complex media have to be applied: Rahse, W. (2012) (“Enzymes for Detergents.” Chemie lngenieur Technik 84(12): 2152-2163) states that industrial production of subtilisin proteases with Bacillus is based on protein rich fermentation media and Maurer, K. H. (2004) (“Detergent proteases.” Current Opinion in Biotechnology 15(4): 330-334) explains that industrial fermentations with Bacillus “are often based on complex, inexpensive nitrogen sources”. Maksym, L. (2010) (Industrielle Fermentation von Bacillus licheniformis zur Produktion von Proteasen) argues that readily available media components like glucose and ammonia repress protease production in Bacillus species. Therefore, complex media components must be used. The nutrients from the complex media components are metabolized slowly because they must be enzymatically released before they are available for the cells. This avoids catabolite repression. Maksym concludes that protein production based on complex raw materials results in a multiple times higher productivity than protein production with defined media. Also, Schuegerl, K. (2004) (“Prozessentwicklung in der Biotechnologie—Ein Rueckblick.” Chemie lngenieur Technik 76(7): 989-1003) reports that they found very low productivities with defined media. They argue that regulatory effects are a dominant factor for the need for complex raw materials for protein production with Bacillus. Ammonia represses protease production while protein can be used beneficially as nitrogen source and corn steep liquor was found to improve product formation due to growth factors that also influence productivity. Further, Huebner, U., U. Bock and K. Schuegerl (1993) (“Production of alkaline serine protease subtilisin Carlsberg by Bacillus licheniformis on complex medium in a stirred tank reactor.” Applied Microbiology and Biotechnology 40(2): 182-188) compared the performance of complex vs. defined mineral media for production of alkaline serine protease subtilisin by Bacillus licheniformis under control of the native promoter of the aprE gene and found that productivity in complex media was significantly superior to chemically defined media (by a factor of up to 1000), concluding that chemically defined media would not be suitable for the production of protease with Bacillus.

In a further study related to the production of amylase in Bacillus subtilis under control of the aprE promoter a fed-batch cultivation based on complex substrates was chosen for high amylase productivity (Chen, J., Y. Gai, G. Fu, W. Zhou, D. Zhang, and J. Wen. 2015. Enhanced extracellular production of alpha-amylase in Bacillus subtilis by optimization of regulatory elements and over-expression of PrsA lipoprotein. Biotechnol. Lett. 37: 899-906).

An example of an established industrial-scale subtilisin production process based on complex media is given by Kueppers, T., V. Steffen, H. Hellmuth, T. O'Connell, J. Bongaerts, K. H. Maurer and W. Wiechert (2014) (“Developing a new production host from a blueprint: Bacillus pumilus as an industrial enzyme producer.” Microbial Cell Factories 13(1): 46) in which both the aprE promoter from Bacillus licheniformis ATCC 53926 as well as the promoters of the aprE1 and aprE2 genes of Bacillus pumilus Jo2 DSM14395 have been used.

The aprE gene of Bacillus encodes for the extracellular protease subtilisin, a valuable enzyme product of biotechnology industry (Marcus Schallmey, Ajay Singh, Owen P Ward, 2004, Developments in the use of Bacillus species for industrial production, Canadian Journal of Microbiology, 2004, 50:1-17). The aprE gene of Bacillus subtilis and the regulation of its expression have been extensively studied.

Inducer-independent promoters, like the aprE promoter, are frequently used for the heterologous expression of proteins in Bacillus, but protein production in industrial-scale has not been successful with such promoters using chemically defined fermentation media.

Wenzel, M., Müller, A., Siemann-Herzberg, M., and Altenbuchner, J. (2011) (“Self-inducible Bacillus subitilis expression system for reliable and inexpensive protein production by high-celldensity fermentation”, Applied and Environmental Microbiology, 77(18), p. 6419-6425) obtained high protein titers of the green fluorescent protein with fermentation of Bacillus subtilis in a chemically defined fermentation medium by modifying the mannose inducible expression system of the mannose operon to make it independent from mannose as inducer and dependent on derepression under glucose limiting conditions. However, in order to obtain an inducer-independent, functional expression system based on the inducer-dependent PmanP promoter adaptations in the mannose metabolism of the Bacillus subtilis cells were necessary, i.e., the deletion of the manA and manP genes of the Bacillus subtilis cells, which codes for the 6-phosphate isomerase and the phosphotransferase system, respectively.

Hence, industrial application of protein production using chemically defined media for Bacillus sp. with standard inducer-independent promoter systems widely used in protein expression in Bacillus, like the aprE promoter, has not been shown to-date. In fact, up to date it was believed that using standard promoter systems requires the application of complex fermentation media.

Thus, there was a need for a robust, cost-efficient, and easy-to-handle industrial fermentation process for the production of proteins in chemically defined media for Bacillus with an industrially proven inducer-independent promotor system due to the advantages these processes generally have for industrial operation compared to complex media processes.

### BRIEF SUMMARY OF THE INVENTION

As a solution to the above referenced problem, the present invention refers to an industrially relevant fermentation process for cultivating a Bacillus cell in a chemically defined fermentation medium comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions.

Furthermore, the present invention also refers to a method of producing a protein of interest comprising the use of the fermentation process described herein. Moreover, the present invention refers to a method for increasing the titer of a protein of interest in a production process comprising the use of the fermentation process as described herein. Also subject of the present invention is a composition comprising a protein of interest produced by a method comprising the use of the fermentation process described herein.

### BRIEF DESCRIPTION OF THE FIGURES

FIG. 1 shows the development of the protease titer over the time during an industrially relevant fermentation process according to the present invention.

FIG. 2 shows the protease titer at the end of the industrially relevant fermentation process of the present invention.

### DETAILED DESCRIPTION OF THE INVENTION

The present invention may be understood more readily by reference to the following detailed description of the preferred embodiments of the invention and the examples included herein.

### Definitions

Unless otherwise noted, the terms used herein are to be understood according to conventional usage by those of ordinary skill in the relevant art.

It is to be understood that as used in the specification and in the claims, “a” or “an” can mean one or more, depending upon the context in which it is used. Thus, for example, reference to “a cell” can mean that at least one cell can be utilized.

Throughout this application, various publications are referenced. The disclosures of all of these publications and those references cited within those publications in their entireties are hereby incorporated by reference into this application in order to more fully describe the state of the art to which this invention pertains.

The term “industrial fermentation” or “industrially relevant fermentation” refers to a fermentation process in which at least 200 g carbon source per liter of initial fermentation medium is added.

A “fermentation process” describes a sequence of activities comprising the preparation of the fermentation medium and the cultivation of cells in the fermentation medium. “Cultivation of the cells” or “growth of the cells” is not understood to be limited to an exponential growth phase with a high rate of cell division but can also include the physiological state of the cells at the beginning of growth after inoculation and during a stationary phase. The fermentation process can be stopped by appropriate measures that limit or prevent the growth of the cells, for instance but not being limited to reducing the temperature of the fermentation broth.

The term “fermentation medium” refers to a water-based solution containing one or more chemical compounds that can support growth of cells.

The term “chemically defined fermentation medium” (also called herein “chemically defined medium”, “defined medium”, or “synthetic medium”) is understood to be used for fermentation media which are essentially composed of chemically defined components in known concentrations. A “chemically defined component” is a component which is known by its chemical formula. A fermentation medium which is essentially composed of chemically defined component includes a medium which does not contain a complex nutrient source, in particular no complex carbon and/or nitrogen source, i.e., which does not contain complex raw materials having a chemically undefined composition. A fermentation medium which is essentially composed of chemically defined components may further include a medium which comprises an essentially small amount of a complex nutrient source, for instance a complex nitrogen and/or carbon source, an amount as defined below, which typically is not sufficient to maintain growth of the microorganism and/or to guarantee formation of a sufficient amount of biomass.

In that regard, complex raw materials have a chemically undefined composition due to the fact that, for instance, these raw materials contain many different compounds, among which complex heteropolymeric compounds, and have a variable composition due to seasonal variation and differences in geographical origin. Typical examples of complex raw materials functioning as a complex carbon and/or nitrogen source in fermentation are soybean meal, cotton seed meal, corn steep liquor, yeast extract, casein hydrolysate, molasses, and the like.

An essentially small amount of a complex carbon and/or nitrogen source may be present in the chemically defined medium according to the invention, for instance as carry-over from the inoculum for the main fermentation. The inoculum for the main fermentation is not necessarily obtained by fermentation on a chemically defined medium. Most often, carry-over from the inoculum will be detectable through the presence of a small amount of a complex nitrogen source in the chemically defined medium of the main fermentation. Small amounts of a complex medium components, like complex carbon and/or nitrogen source, might also be introduced into the fermentation medium by the addition of small amounts of these complex components to the fermentation medium. It may be advantageous to use a complex carbon and/or nitrogen source in the fermentation process of the inoculum for the main fermentation, for instance to speed up the formation of biomass. i.e. to increase the growth rate of the microorganism, and/or to facilitate internal pH control. For the same reason, it may be advantageous to add an essentially small amount of a complex carbon and/or nitrogen source, e.g. yeast extract, to the initial stage of the main fermentation, especially to speed up biomass formation in the early stage of the fermentation process.

An essentially small amount of a complex nutrient source which may be added to the fermentation medium in the fermentation process according to the invention is defined to be an amount of at the most 10% of the total amount of the respective nutrient, which is added in the fermentation process. In particular, an essentially small amount of a complex carbon and/or nitrogen source which may be added to the fermentation medium in the fermentation process according to the invention is defined to be an amount of a complex carbon source resulting in at the most 10% of the total amount of carbon and/or an amount of a complex nitrogen source resulting in at the most 10% of the total amount of nitrogen, which is added in the fermentation process, preferably an amount of a complex carbon source resulting in at the most 5% of the total amount of carbon and/or an amount of a complex nitrogen source resulting in at the most 5% of the total amount of nitrogen, more preferably an amount of a complex carbon source resulting in at the most 1% of the total amount of carbon and/or an amount of a complex nitrogen source resulting in at the most 1% of the total amount of nitrogen, which is added in the fermentation process. Preferably, at the most 10% of the total amount of carbon and/or at the most 10% of the total amount of nitrogen, preferably an amount of at the most 5% of the total amount of carbon and/or an amount of at the most 5% of the total amount of nitrogen, more preferably an amount of at the most 1% of the total amount of carbon and/or an amount of at the most 1% of the total amount of nitrogen which is added in the fermentation process is added via carry-over from the inoculum. Most preferably, no complex carbon and/or complex nitrogen source is added to the fermentation medium in the fermentation process.

It is to be understood that the term “chemically defined fermentation medium” as used in the present invention includes a medium wherein, except for the fed chemically defined carbon source and the fed chemically defined magnesium ion source, all components are added to the medium before inoculation with Bacillus cells, and further includes a medium wherein part of the components are added before and parts are added to the medium after inoculation, preferably, as one or more feed solutions.

The term “initial chemically defined fermentation medium” or “initial fermentation medium” or “initial medium” refers to the fermentation medium prior inoculation with the cell. Thus, the initial chemically defined fermentation medium can either comprise, except for the fed chemically defined carbon source and the fed chemically defined magnesium ion source, all nutrient sources added during the fermentation process or only a part of the nutrient sources added during the fermentation process, wherein in case of the latter the remaining parts are added after inoculation with cells.

The term “chemically defined nutrient source” (e.g., “chemically defined carbon source” or “chemically defined nitrogen source”) is understood to be used for nutrient sources which are composed of chemically defined compounds.

The term “fermentation broth” refers to the fermentation medium comprising the cells. Hence, the term “added to the fermentation medium during the cultivation of the cells” refers to the addition of components to the fermentation medium comprising cells, i.e., to the fermentation broth.

The term “feed solution” is used herein for a solution that is added during the fermentation process to the fermentation medium after inoculation of the initial fermentation medium with the cell, which comprises compounds supportive for the growth of the cells. It is understood herein that at least part of the compounds that are provided as feed solution can already be present to a certain extend in the fermentation medium prior the feeding of said compounds. Various feed profiles are known in the art. A feed solution can be added continuously or discontinuously during the fermentation process. Discontinuous addition of a feed solution can occur once during the fermentation process as a single bolus or several times at various or same volumes. Continuous addition of a feed solution can occur during the fermentation process at the same or at varying rates (i.e., volume per time). Also combinations of continuous and discontinuous feeding profiles can be applied during the fermentation process. Components of the fermentation medium that are provided as feed solution can be added in one feed solution or as different feed solutions. In case more than one feed solutions are applied, the feed solutions can have the same or different feed profiles as described above. Preferably, the one or more feed solutions are provided throughout the fermentation process either as continuous feed or as several separate bolus additions at various or at same volumes.

“Trace elements” as used herein are elements taken from the list of iron, copper, manganese, zinc, cobalt, nickel, molybdenum, selenium, and boron.

The term “titer of a protein of interest” as used herein is understood as the amount of protein of interest in g per volume of fermentation broth in liter.

The term “added in the fermentation process” or “added during the fermentation process” regarding the amount of a certain compound of the fermentation medium describes the total amount of the compound added during the fermentation process, i.e., including an amount of the compound in the initial fermentation medium as well as an amount added during the cultivation of the cells by means of one or more feed solutions.

For the present invention “the addition of one or more feed solutions comprising one or more chemically defined carbon sources and magnesium ions to the fermentation medium” shall be understood in a way that chemically defined carbon sources and magnesium ions are added to the fermentation medium after inoculation, i.e., to the fermentation broth, in the same feed solution or by separate feed solutions or combinations thereof. One or more different sources of carbon or one or more different sources of magnesium ions can be added to the fermentation medium with the same or with different feed solutions.

The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively.

“Parent” sequence (e.g., “parent enzyme” or “parent protein”) is the starting sequences for introduction of changes (e.g. by introducing one or more amino acid substitutions) of the sequence resulting in “variants” of the parent sequences. Thus, the term “enzyme variant” or “sequence variant” or “protein variant” are used in reference to parent enzymes that are the origin for the respective variant enzymes. Therefore, parent enzymes include wild type enzymes and variants of wild-type enzymes which are used for development of further variants. Variant enzymes differ from parent enzymes in their amino acid sequence to a certain extent; however, variants at least maintain the enzyme properties of the respective parent enzyme. In one embodiment, enzyme properties are improved in variant enzymes when compared to the respective parent enzyme. In one embodiment, variant enzymes have at least the same enzymatic activity when compared to the respective parent enzyme or variant enzymes have increased enzymatic activity when compared to the respective parent enzyme.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percentidentity applies:

%-identity=(identical residues/length of the alignment region which is showing the respective sequence of this invention over its complete length)*100. Thus sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications:

For DNA sequences encoding for a protein the pairwise alignment shall be made over the complete length of the coding region from start to stop codon excluding introns.

For non-protein-coding DNA sequences the pairwise alignment shall be made over the complete length of the sequence of this invention, so the complete sequence of this invention is compared to another sequence, or regions out of another sequence.

Moreover, for DNA sequences the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

For the promoter sequences of this invention, the sequence identity with any other sequence shall be calculated as follows:

In a first step, the promoter sequence of this invention shall be aligned with a second sequence by a local alignment, for example using programs Blast (NCBI, nucleotide default settings) or Water (EMBOSS, nucleotide default settings). Only local alignments, in which at least 190 bases of the promoter sequence of this invention are comprised by the alignment, are considered and are used to calculate identity. The %-identity is then calculated as: %-identity=(identical residues/length of the local alignment). This value is multiplied with 100 to give “%-identity”.

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques. With respect to two or more polynucleotide sequences or two or more amino acid sequences, the term “heterologous” is used to characterized that the two or more polynucleotide sequences or two or more amino acid sequences are naturally not occurring in the specific combination with each other.

For the purposes of the invention, “recombinant” (or transgenic) with regard to a cell or an organism means that the cell or organism contains a heterologous polynucleotide which is introduced by man by gene technology and with regard to a polynucleotide includes all those constructions brought about by man by gene technology/recombinant DNA techniques in which either

(a) the sequence of the polynucleotide or a part thereof, or

(b) one or more genetic control sequences which are operably linked with the polynucleotide, including but not limited thereto a promoter, or

(c) both a) and b) are not located in their wildtype genetic environment or have been modified.

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic. The term “nucleic acid construct” is synonymous with the term “expression cassette” when the nucleic acid construct contains the control sequences required for expression of a polynucleotide.

The term “control sequence” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS, shine dalgarno sequence), 3′-UTR, and transcription start and stop sites.

The term “functional linkage” or “operably linked” with respect to regulatory elements, is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. Promoter is followed by the transcription start site of the gene. Promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule which presence in the fermentation medium is capable of affecting an increase in expression of a gene by increasing the activity of an inducer-dependent promoter operably linked to the gene. Preferably the inducer molecule is a carbohydrate or an analog thereof. In one embodiment, the inducer molecule is a secondary carbon source of the Bacillus cell. In the presence of a mixture of carbohydrates cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β-gluco- | B. subtilis |
| | | | sides | |
| levDEFG | LevR | A | fructose | B. subtilis |
| sacL | | | | |
| mtlAD | MtlR | A | mannitol | B. subtilis |
| manPA-yjdF | ManR | A | mannose | B. subtilis |
| manR | ManR | A | mannose | B. subtilis |
| bglFB bglG | BglG | AT | β-glucosides | E. coli |
| lacTEGF | LacT | AT | lactose | L. casei |
| lacZYA | lacI | R | Allolactose; | E. coli |
| | | | IPTG (Isopropyl | |
| | | | β-D-1-thiogalac- | |
| | | | topyranoside) | |
| araBAD | araC | AR | L-arabinose | E. coli |
| xylAB | XylR | R | Xylose | B. subtilis |

a): transcriptional regulator protein
b): A: activator
AT: antiterminator
R: repressor
AR: activator/repressor

In contrast thereto, the activity of promoters that do not depend on the presence of an inducer molecule added to the fermentation medium (herein called “inducer-independent promoters”) are either constitutively active or can be increased regardless of the presence of an inducer molecule that is added to the fermentation medium.

In a preferred embodiment the inducer-independent promoter is an aprE promoter.

An “aprE promoter” or “aprE promoter sequence” is the nucleotide sequence (or parts or variants thereof) located upstream of an aprE gene, i.e., a gene coding for a Bacillus subtilisin Carlsberg protease, on the same strand as the aprE gene that enables that aprE gene's transcription.

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

The term “expression vector” is defined herein as a linear or circular DNA molecule that comprises a polynucleotide that is operably linked to one or more control sequences that provides for the expression of the polynucleotide.

The term “host cell”, as used herein, includes any cell type that is susceptible to transformation, transfection, transduction, conjugation, and the like with a nucleic acid construct or expression vector.

The term “introduction of DNA into a cell” and variations thereof are defined herein as the transfer of a DNA into a host cell. The introduction of a DNA into a host cell can be accomplished by any method known in the art, including, the not limited to, transformation, transfection, transduction, conjugation, and the like.

The term “donor cell” is defined herein as a cell that is the source of DNA introduced by any means to another cell.

The term “recipient cell” is defined herein as a cell into which DNA is introduced.

The “HMM-score” is the score value obtained by the method used in Example 2.

### DETAILED DESCRIPTION

The present invention is directed to an industrially relevant fermentation process for producing a protein of interest in Bacillus cells using a chemically defined fermentation medium. The fermentation process described herein extends the scope of usual lab scale fermentation. In particular, the inventors of the present invention revealed that feeding magnesium ions—usually provided in industrially relevant fermentation in the batch medium—during cultivation of the Bacillus cells to a chemically defined fermentation medium produces biomass and protein yields with industrially relevant titers. Thus, in one embodiment the present invention provides a fermentation process for cultivating a Bacillus cell in a chemically defined fermentation medium comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions.

Chemically Defined Fermentation Medium

Culturing a microorganism in a chemically defined fermentation medium requires that cells be cultured in a medium which contain various chemically defined nutrient sources selected from the group consisting of chemically defined hydrogen source, chemically defined oxygen source, chemically defined carbon source, chemically defined nitrogen source, chemically defined sulfur source, chemically defined phosphorus source, chemically defined magnesium source, chemically defined sodium source, chemically defined potassium source, chemically defined trace element source, and chemically defined vitamin source. Unless marked otherwise, within this description, nutrient sources used to prepare the chemically defined fermentation medium shall be understood as being chemically defined nutrient sources even if not explicitly mentioned.

Preferably, the chemically defined carbon source is selected from the group consisting of carbohydrates, organic acids, hydrocarbons, and alcohols and mixtures thereof. Preferred carbohydrates are selected from the group consisting of glucose, fructose, galactose, xylose, arabinose, sucrose, maltose, maltotriose, lactose, dextrin, maltodextrins, starch and inulin, and mixtures thereof. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Preferably, the chemically defined carbon source comprises glucose or sucrose. More preferably, the chemically defined carbon source comprises glucose, preferably wherein the predominant amount of the chemically defined carbon source is provided as glucose. Most preferably, the chemically defined carbon source is glucose. It is to be understood that the chemically defined carbon source can be provided in form of a syrup, preferably as glucose syrup. As understood herein, the term “glucose” shall include glucose syrups. A glucose syrup is a viscous sugar solution with high sugar concentration. The sugars in glucose syrup are mainly glucose and to a minor extend also maltose and maltotriose in varying concentrations depending on the quality grade of the syrup. Preferably, besides glucose, maltose and maltotriose the syrup can comprise up to 10%, preferably, up to 5%, more preferably up to 3% impurities. Preferably, the syrup is corn syrup.

The chemically defined nitrogen source is preferably selected from the group consisting of urea, ammonia, nitrate, nitrate salts, nitrit, ammonium salts such as ammonium chloride, ammonium sulphate, ammonium acetate, ammonium phosphate and ammonium nitrate, and amino acids such as glutamate or lysine and combinations thereof. More preferably, a chemically defined nitrogen source is selected from the group consisting of ammonia, ammonium sulphate and ammonium phosphate. Most preferably, the chemically defined nitrogen source is ammonia. The use of ammonia as a chemically defined nitrogen source has the advantage that ammonia can additionally function as a pH controlling agent. Preferably, at least 0.1 g of nitrogen is added per liter of initial fermentation medium in the initial fermentation medium.

Oxygen is usually provided during the cultivation of the cells by aeration of the fermentation media by stirring or gassing. Hydrogen is usually provided due to the presence of water in the aqueous fermentation medium. However, hydrogen and oxygen are also contained within the chemically defined carbon and/or chemically defined nitrogen source and can be provided that way.

Magnesium can be provided to the fermentation medium in chemically defined form by one or more magnesium salts, preferably one or more selected from the group consisting of magnesium chloride, magnesium sulfate, magnesium nitrate, and magnesium phosphate, or by magnesium hydroxide, or by combinations of one or more magnesium salts and magnesium hydroxide. In addition to the magnesium provided via one or more feed solutions additional magnesium can be provided in the initial fermentation medium.

Sodium can be added to the fermentation medium in chemically defined form by one or more sodium salts, preferably selected from the group consisting of sodium chloride, sodium nitrate, sodium sulphate, sodium phosphate, sodium hydroxide, and combinations thereof. Preferably, at least 0.1 g of sodium is added per liter of initial fermentation medium in the initial fermentation medium.

Calcium can be added to the fermentation medium by one or more calcium salts, preferably selected from the group consisting of calcium sulphate, calcium chloride, calcium nitrate, calcium phosphate, calcium hydroxide, and combination thereof. Preferably, at least 0.01 g of calcium is added per liter of initial fermentation medium in the initial fermentation medium.

Potassium can be added to the fermentation medium in chemically defined form by one or more potassium salts, preferably selected from the group consisting of potassium chloride, potassium nitrate, potassium sulphate, potassium phosphate, potassium hydroxide, and combination thereof. Preferably, at least 0.4 g of potassium is added per liter of initial fermentation medium in the initial fermentation medium.

Phosphorus can be added to the fermentation medium in chemically defined form by one or more salts comprising phosphorus, preferably selected from the group consisting of potassium phosphate, sodium phosphate, magnesium phosphate, phosphoric acid, and combinations thereof. Preferably, at least 1 g of phosphorus is added per liter of initial fermentation medium in the initial fermentation medium.

Sulfur can be added to the fermentation medium in chemically defined form by one or more salts comprising sulfur, preferably selected from the group consisting of potassium sulfate, sodium sulfate, magnesium sulfate, sulfuric acid, and combinations thereof. Preferably, at least 0.15 g of sulfur is added per liter of initial fermentation medium in the initial fermentation medium.

Preferably, the initial chemically defined fermentation medium comprises one or more selected from the group consisting of:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium;

Preferably, the initial chemically defined fermentation medium comprises one or more selected from the group consisting of:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium;

0.01-3 g calcium per liter of initial fermentation medium;

50 μmol to 5 mmol per liter of initial medium iron;

40 μmol to 4 mmol per liter of initial medium copper;

30 μmol to 3 mmol per liter of initial medium manganese;

40 μmol to 2 mmol per liter of initial medium zinc;

1 μmol to 100 μmol per liter of initial medium cobalt;

2 μmol to 200 μmol per liter of initial medium nickel; and

0.3 μmol to 50 μmol per liter of initial medium molybdenum.

More preferably, the initial chemically defined fermentation medium comprises:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium.

More preferably, the initial chemically defined fermentation medium comprises:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium; and

optionally one or more selected from the group consisting of

50 μmol to 5 mmol per liter of initial medium iron;

40 μmol to 4 mmol per liter of initial medium copper;

30 μmol to 3 mmol per liter of initial medium manganese, and

40 μmol to 2 mmol per liter of initial medium zinc, and

optionally one or more selected from the group consisting of

1 μmol to 100 μmol per liter of initial medium cobalt;

2 μmol to 200 μmol per liter of initial medium nickel; and

0.3 μmol to 50 μmol per liter of initial medium molybdenum.

In addition to the magnesium ions provided via one or more feed solutions additional magnesium ions can be added to the initial fermentation medium in chemically defined form. Preferably, the initial chemically defined fermentation medium comprises:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium; and

optionally 0.1-10 g magnesium per liter of initial fermentation medium.

In addition to the magnesium ions provided via one or more feed solutions additional magnesium ions can be added to the initial fermentation medium in chemically defined form. Preferably, the initial chemically defined fermentation medium comprises:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium; and

optionally 0.1-10 g magnesium per liter of initial fermentation medium; and

optionally one or more selected from the group consisting of

50 μmol to 5 mmol per liter of initial medium iron;

40 μmol to 4 mmol per liter of initial medium copper;

30 μmol to 3 mmol per liter of initial medium manganese, and

40 μmol to 2 mmol per liter of initial medium zinc, and

optionally one or more selected from the group consisting of

1 μmol to 100 μmol per liter of initial medium cobalt;

2 μmol to 200 μmol per liter of initial medium nickel; and

0.3 μmol to 50 μmol per liter of initial medium molybdenum.

One or more trace element ions can be added to the fermentation medium in chemically defined form. These trace element ions are selected from the group consisting of iron, copper, manganese, and zinc. Also one or more trace elements selected from cobalt, nickel, molybdenum, selenium, and boron can be added. Preferably, the trace element ions iron, copper, manganese, and zinc are added, and optionally one or more selected from cobalt, nickel, and molybdenum are added to the fermentation medium. Preferably, the one or more trace element ions are added to the initial fermentation medium in an amount selected from the group consisting of at least 50 μmol per liter of initial medium iron, at least 40 μmol per liter of initial medium copper, at least 30 μmol per liter of initial medium manganese, at least 40 μmol per liter of initial medium zinc, at least 1 μmol per liter of initial medium cobalt, at least 2 μmol per liter of initial medium nickel, and at least 0.3 μmol per liter of initial medium molybdenum. Preferably, the one or more trace element ions are added to the initial fermentation medium in an amount selected from the group consisting of 50 μmol to 5 mmol per liter of initial medium iron, 40 μmol to 4 mmol per liter of initial medium copper, 30 μmol to 3 mmol per liter of initial medium manganese, 40 μmol to 2 mmol per liter of initial medium zinc, 1 μmol to 100 μmol per liter of initial medium cobalt, 2 μmol to 200 μmol per liter of initial medium nickel, and 0.3 μmol to 50 μmol per liter of initial medium molybdenum. For adding each trace element preferably one or more from the group consisting of chloride, phosphate, sulphate, nitrate, citrate and acetate salts can be used.

Compounds which may optionally be included in a chemically defined medium are chelating agents, such as citric acid, MGDA, NTA, or GLDA, and buffering agents such as mono- and dipotassium phosphate, calcium carbonate, and the like. Preferably, the chemically defined fermentation medium comprises citric acid. Buffering agents preferably are added when dealing with processes without an external pH control. In addition, an antifoaming agent may be dosed prior to and/or during the fermentation process.

The chemically defined medium may also comprise vitamins. Vitamins refer to a group of structurally unrelated organic compounds which are necessary for the normal metabolism of cells. A vitamin should be added to the fermentation medium of Bacillus cells not capable to synthesize said vitamin. Vitamins can be selected from the group of thiamin, riboflavin, pyridoxal, nicotinic acid or nicotinamide, pantothenic acid, cyanocobalamin, folic acid, biotin, lipoic acid, purines, pyrimidines, inositol, choline, and hemins.

Preferably, the fermentation medium also comprises a selection agent, e.g., an antibiotic, such as ampicillin, tetracycline, kanamycin, hygromycin, bleomycin, chloroamphenicol, streptomycin or phleomycin, to which the selectable marker of the cells provides resistance.

The amount of necessary compounds to be added to the chemically defined medium will mainly depend on the amount of biomass which is to be formed in the fermentation process. The amount of biomass formed may vary typically from about 10 to about 150 grams of dry cell mass per liter of fermentation broth. Usually, for protein production, fermentation proecesses producing an amount of biomass which is lower than about 10 g of dry cell mass per liter of fermentation broth are not considered industrially relevant.

The optimum amount of each component of a chemically defined medium will depend on the type of Bacillus strain which is subjected to fermentation in a defined medium, on the amount of biomass and on the protein of interest to be formed. The use of chemically defined media thereby advantageously allows for a variation of the concentration of each medium component independently from the other components, in this way facilitating optimization of the medium composition. Typically, the amount of medium components necessary for growth of the Bacillus cell may be determined in relation to the amount of carbon source used in the fermentation, since the amount of biomass formed will be primarily determined by the amount of carbon source used.

An industrially relevant fermentation process preferably encompasses a fermentation process on a volume scale which is at least 1 m3 with regard to the nominal fermenter size, preferably at least 5 m3, more preferably at least 10 m3, even more preferably at least 25 m3, most preferably at least 50 m3. Preferably, the industrially relevant fermentation process encompasses a fermentation process on a volume scale which is 1-500 m3 with regard to the nominal fermenter size, preferably 5-500 m3, more preferably 10-500 m3, even more preferably 25-500 m3, most preferably 50-500 m3.

Preferably, prior inoculation the chemically defined medium and feed solutions are sterilized in order to prevent or reduce growth of microorganisms during the fermentation process, which are different from the inoculated Bacillus cells. Sterilization can be performed with methods known in the art, for example but not limited to autoclaving or sterile filtration. Medium components can be sterilized separately from other medium components to avoid interactions of medium components during sterilization treatment or to avoid decomposition of medium components under certain sterilization conditions.

Preferably, the pH of the chemically defined medium is adjusted prior to inoculation. Preferably, the pH of the chemically defined medium is adjusted prior to inoculation, but after sterilization. Preferably, the pH of the chemically defined medium is adjusted prior inoculation to pH 6.6 to 9, preferably to pH 6.6 to 8.5, more preferably to pH 6.8 to 8.5, most preferably to pH 6.8 to pH 8.0.

Fermentation Process

As described above, the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, i.e., to the fermentation broth, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions.

The fermentation process of the present invention comprises the steps of preparing the initial fermentation medium as described above, the inoculation of the fermentation medium with the Bacillus cell and the cultivation of the Bacillus cell in the fermentation medium. Optionally, prior inoculation of the initial chemically defined fermentation medium with the Bacillus cell the initial chemically defined fermentation medium is sterilized and optionally the initial pH is set.

Thus, in a first step, a chemically defined fermentation medium as described herein is prepared. The fermentation medium then is preferably sterilized with methods known in the art in order to prevent or reduce the growth of microorganisms during the fermentation process that differ from the microorganisms inoculated into the fermentation medium.

Inoculation of the chemically defined fermentation medium with the Bacillus cells can be done by inoculation with or without a starter culture (pre-culture). Preferably, the fermentation is inoculated with a pre-culture that has been grown under conditions known to the person skilled in the art. The pre-culture can be obtained by cultivating the cells in a chemically defined pre-culture medium or in a complex pre-culture medium. The chemically defined pre-culture medium can be the same or different to the chemically defined fermentation medium used during the main fermentation process. The complex pre-culture medium can contain complex nitrogen and/or complex carbon sources. Preferably, the pre-culture is obtained by using a complex culture medium. The pre-culture broth can be added all or in part to the main fermentation medium. The volume ratio between pre-culture broth used for inoculation and main fermentation medium is preferably 0.1-30%.

The main fermentation process of the present invention is a fed-batch process. In a fed-batch process, only a part of the compounds of the chemically defined fermentation medium used in the fermentation process is added to the fermentation medium before inoculation of the fermentation medium with cells and the start of the fermentation and the remaining part of the compounds is added during the fermentation process. According to the present invention at least parts of the chemically defined carbon source and at least parts of the magnesium ions are fed to the fermentation medium during cultivation of the cells. In a specific embodiment, the fermentation process of the present invention can be realized as a repeated fed-batch process or continuous fermentation process. In a repeated fed-batch or continuous fermentation process, the complete start medium is additionally fed during fermentation. The start medium can be fed together with or separate from the other feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth.

The chemically defined compounds comprising a particular nutrient source selected for feeding can be the same or different to the chemically defined compounds comprising this particular nutrient source provided in the initial fermentation medium.

The chemically defined compounds which are selected for feeding to the fermentation medium can be fed together in one feed solution or separate from each other in different feed solutions and combinations thereof. The compounds that are added during the cultivation of the cells can in part be already present in the batch medium. A feed solution can be added continuously or discontinuously during the fermentation process. Discontinuous addition of a feed solution can occur once during the fermentation process as a single bolus or several times at various or same volumes. Continuous addition of a feed solution can occur during the fermentation process at the same or at varying rates (i.e., volume per time). Also combinations of continuous and discontinuous feeding profiles can be applied during the fermentation process. Preferably, one or more feeding solutions are added continuously. Components of the fermentation medium that are provided as feed solution can be added in one feed solution or as different feed solutions. In case more than one feed solutions are applied, the feed solutions can have the same or different feed profiles as described above. Preferably, the one or more feed solutions are provided throughout the fermentation process either as continuous feed or as several separate bolus additions at various or at same volumes.

In the fermentation process of the present invention, at least the one or more chemically defined carbon sources and the one or more chemically defined sources of magnesium ions are provided at least in parts as feed solutions. This allows to obtain high protein yields under industrially relevant fermentation conditions using a chemically defined fermentation medium. Chemically defined carbon source and magnesium ions can be added in one or in more than one feed solutions, the latter with the chemically defined carbon source and magnesium ions being present in separated feed solutions. Preferably, the chemically defined carbon source and magnesium ions are added with separate feed solutions. In a preferred embodiment of the invention, also the chemically defined nitrogen source and/or sulfur source and/or the phosphorus source and/or trace element source or at least parts thereof are fed to the fermentation process. In a more preferred embodiment, the chemically defined carbon and chemically defined nitrogen source and the chemically defined magnesium ion source are fed to the fermentation process.

In a further preferred embodiment, the chemically defined carbon source and chemically defined trace element source (preferably one or more selected from Fe, Cu, Mn, and Zn, and optionally in addition one or more selected from Co, Ni, and Mo, more preferably all of Fe, Cu, Mn, and Zn, and optionally in addition one or more selected from Co, Ni, and Mo) and the chemically defined magnesium ion source or at least parts thereof are fed to the fermentation process. This allows to obtain high protein yields under industrially relevant fermentation conditions using a chemically defined fermentation medium. In a more preferred embodiment, the chemically defined carbon source and chemically defined trace element source and chemically defined nitrogen source and the chemically defined magnesium ion source or at least parts thereof are fed to the fermentation process. In a more preferred embodiment, the chemically defined carbon source and chemically defined trace element source and chemically defined nitrogen source and the chemically defined magnesium ion source or at least parts thereof as well as the chemically defined sulfur source or at least parts thereof are fed to the fermentation process. Further preferred, the chemically defined carbon and chemically defined nitrogen source and chemically defined magnesium ion source, as well as chemically defined sulfur and chemically defined phosphorus source or at least parts thereof are fed. In a more preferred embodiment, the chemically defined carbon source and chemically defined trace element source and chemically defined nitrogen source and the chemically defined magnesium ion source or at least parts thereof as well as chemically defined sulfur source and phosphorous source or at least parts thereof are fed to the fermentation process.

Chemically defined carbon source, trace element ions, and magnesium ions can be added in one or in more than one feed solutions, the latter with the chemically defined carbon source, trace element ions, and the magnesium ions being present in separated feed solutions. Preferably, the chemically defined carbon source, trace element ions, and magnesium ions are added with separate feed solutions. Preferably, the chemically defined nitrogen source is added as an additional separate feed solution. The different trace elements can be added with one single feed or with separate feed solutions. Preferably, the different trace element ions are added with one single feed solution.

In that regard, a preferred chemically defined carbon source is glucose and a preferred chemically defined nitrogen source is ammonia and/or ammonium salts. Preferred magnesium source is magnesium sulfate.

Preferably, at least 50% of the chemically defined carbon source and at least 50% of the magnesium ions is provided in the fermentation process as feed solution. In one embodiment, at least 50% of the chemically defined carbon source, at least 50% of chemically defined nitrogen source, and at least 50% of the magnesium ions is provided in the fermentation process as feed solution. In one embodiment, at least 50% of the chemically defined carbon source, at least 50% of the trace element ions, and at least 50% of the magnesium ions is provided in the fermentation process as feed solution. In one embodiment, at least 50% of the chemically defined carbon source, at least 50% of the trace element ions, at least 50% of the magnesium ions, and at least 50% of the chemically defined nitrogen source is provided in the fermentation process as feed solution. In one embodiment, at least 50% of the chemically defined carbon source, at least 50% of the trace element ions, at least 50% of the magnesium ions, at least 50% of the chemically defined nitrogen source, and at least 50% of the chemically defined sulfur source is provided in the fermentation process as feed solution. In one embodiment, at least 50% of the chemically defined carbon source, at least 50% of the trace element ions, at least 50% of the magnesium ions, at least 50% of the chemically defined nitrogen source, at least 50% of the chemically defined sulfur source, and at least 50% of the chemically defined phosphorus source is provided in the fermentation process as feed solution.

Preferably, at least 60%, at least 70%, at least 80%, at least 90%, or 100% of the chemically defined carbon source provided in the fermentation process is provided as feed solution to the fermentation process. More preferably, at least 90% or 100% of the chemically defined carbon source provided in the fermentation process is provided as feed solution to the fermentation process.

Preferably, at least 60%, at least 70%, at least 80%, at least 90%, or 100% of the magnesium ions provided in the fermentation process is provided as feed solution to the fermentation process. More preferably, at least 90% or 100% of the magnesium ions provided in the fermentation process is provided as feed solution to the fermentation process.

Preferably, at least 60%, at least 70%, at least 80%, at least 90%, or 100% of the trace element ions provided in the fermentation process is provided as feed solution to the fermentation process. More preferably, at least 90% or 100% of the trace element ions provided in the fermentation process is provided as feed solution to the fermentation process.

Preferably, at least 60%, at least 70%, at least 80%, at least 90%, or 100% of the chemically defined nitrogen source provided in the fermentation process is provided as feed solution to the fermentation process. More preferably, at least 90% or 100% of the chemically defined nitrogen source is provided as feed solution to the fermentation process.

Preferably, at least 60%, at least 70%, at least 80%, at least 90%, or 100% of the chemically defined sulfur source provided in the fermentation process is provided as feed solution to the fermentation process.

Preferably, at least 60%, at least 70%, at least 80%, at least 90%, or 100% of the chemically defined phosphorus source provided in the fermentation process is provided as feed solution to the fermentation process.

Most preferably, at least 90% or 100% of the chemically defined carbon source, at least 90% or 100% of the magnesium ions, and at least 90% or 100% of the chemically defined nitrogen source provided in the fermentation process is provided as feed solution to the fermentation process, preferably, in addition, at least 90% or 100% of the trace element ions provided in the fermentation process is provided as feed solution to the fermentation process. Preferably, at least 90% of the chemically defined carbon source and at least 90% of the magnesium provided in the fermentation process is provided as feed solution to the fermentation process. Preferably, at least 90% of the chemically defined carbon source, at least 90% of the magnesium ions, and at least 90% of the chemically defined nitrogen source provided in the fermentation process is provided as feed solution to the fermentation process, preferably, in addition, at least 90% of the trace element ions provided in the fermentation process is provided as feed solution to the fermentation process.

The use of a fed-batch process typically enables the use of a considerably higher amount of chemically defined carbon and chemically defined nitrogen source than is used in a batch process. Specifically, the amount of chemically defined carbon and chemically defined nitrogen source applied in a fed-batch process can be at least about two times higher than the highest amount applied in a batch process. This, in turn, leads to a considerably higher amount of biomass formed in a fed-batch process.

In the fermentation process of the present invention, one or more feed solutions comprising one or more chemically defined carbon sources are added to the fermentation broth. Preferably, the one or more chemically defined carbon source feeding solutions are added continuously. The total amount of chemically defined carbon source, preferably glucose, added in the fermentation process is above 200 g of carbon source per liter of initial fermentation medium. Preferably, the total amount of chemically defined carbon source added in the fermentation process is above 300 g, more preferably above 400 g per liter of initial fermentation medium of carbon source added in the fermentation process. Preferably, at least 50% of the chemically defined carbon source is provided in the fermentation process as feed solution, more preferred at least 60%, at least 70%, at least 80%, more preferred at least 90%, or 100% of the chemically defined carbon source provided in the fermentation process is provided as feed solution in the fermentation process. The feeding of such amounts of chemically defined carbon source allows for the formation of biomass and protein of interest in quantities needed in industrial fermentation processes using a chemically defined fermentation medium.

In the fermentation process of the present invention, one or more feed solutions comprising magnesium ions are added to the fermentation broth during cultivation of the cells. Preferably, the one or more magnesium feeding solutions are added continuously. The inventors of the present invention revealed that adding magnesium as feed solution increases biomass and titer of the protein of interest. By providing a significant amount of magnesium as feed solution the protein titer is significantly improved. At least 0.1 gram magnesium ions per liter of initial fermentation medium is added to the fermentation medium during the cultivation of the Bacillus cell by the one or more feed solutions comprising the magnesium ions. In a preferred embodiment, the magnesium ions and the chemically defined carbon source, which is preferably glucose, are added by separate feed solutions. Preferably, at least 0.3 gram, more preferred at least 0.4 gram of magnesium ions per liter of initial fermentation medium is added to the fermentation medium during the cultivation of the Bacillus cell by the one or more feed solutions comprising the magnesium ions. Preferably, a total of at most 10 g magnesium ions per liter of initial fermentation medium, more preferably at most 5 g magnesium ions per liter of initial fermentation medium, even more preferably at most 2 g magnesium ions per liter of initial fermentation medium, most preferably at most 1 g magnesium ions per liter of initial fermentation medium of magnesium ions are added in the fermentation process. Preferably, magnesium ions in an amount of 0.1-10 g magnesium ions, more preferably 0.3-8 g, even more preferably 0.3-2 g, even more preferably, 0.4-1 g magnesium ions, most preferably 0.4-0.9 g magnesium ions per liter of initial fermentation medium are added to the fermentation medium during the cultivation of the Bacillus cell by the one or more feed solutions comprising the magnesium ions.

Preferably, at least 50% of the magnesium ions is provided in the fermentation process as feed solution, more preferred at least 60%, at least 70%, at least 80%, at least 90%, or 100% of the magnesium cations provided in the fermentation process are provided as feed solution in the fermentation process. More preferred, at least 90% of the magnesium cations provided in the fermentation process are provided as feed solution in the fermentation process.

Preferably, the magnesium ions are provided by one or more magnesium salts, preferably one or more selected from the group consisting of magnesium chloride, magnesium sulfate, magnesium nitrate, and magnesium phosphate, or by magnesium hydroxide, or by combinations of one or more magnesium salts and magnesium hydroxide.

Thus, in a preferred embodiment the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, and
   wherein the total amount of chemically defined carbon source, preferably glucose, added in the fermentation process is above 200 g of carbon source per liter of initial fermentation medium; and
   wherein at least 0.1 gram magnesium ions per liter of initial fermentation medium is added to the fermentation medium during the cultivation of the Bacillus cell by the one or more feed solutions comprising the magnesium ions, wherein at least 50% of the chemically defined carbon source and at least 50% of the magnesium ion source is provided in the fermentation process as feed solution, more preferred at least 60%, at least 70%, at least 80%, most preferably, at least 90%, or 100% of the chemically defined carbon source and at least 60%, at least 70%, at least 80%, most preferably, at least 90%, or 100% of the magnesium ion source provided in the fermentation process is provided as feed solution in the fermentation process.

Preferably, one or more chemically defined nutrient sources are added in the fermentation process comprising one or more selected from the group consisting of:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium.

More preferably, chemically defined nutrient sources are added in the fermentation process comprising:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium.

More preferably, chemically defined nutrient sources are added in the fermentation process comprising:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium; and

optionally one or more selected from the group consisting of

50 μmol to 5 mmol per liter of initial medium iron;

40 μmol to 4 mmol per liter of initial medium copper;

30 μmol to 3 mmol per liter of initial medium manganese, and

40 μmol to 2 mmol per liter of initial medium zinc, and

optionally one or more selected from the group consisting of

1 μmol to 100 μmol per liter of initial medium cobalt;

2 μmol to 200 μmol per liter of initial medium nickel; and

0.3 μmol to 50 μmol per liter of initial medium molybdenum.

In one embodiment, the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, i.e., to the fermentation broth, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions;

wherein chemically defined nutrient sources are added in the fermentation process comprising:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium;

preferably, wherein one or more selected from at least 50% of the nitrogen, at least 50% of the phosphorus, at least 50% of the sulphur, at least 50% of the potassium, at least 50% of the sodium, and at least 50% of the calcium are provided by one or more feed solutions during the cultivation of the cells; preferably wherein at least 50% of the nitrogen and at least 50% of the sulphur is provided by one or more feed solutions during the cultivation of the cells.

In another embodiment, the initial chemically defined fermentation medium comprises one or more chemically defined nutrient sources comprising one or more selected from the group consisting of:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium.

Preferably, the initial chemically defined fermentation medium comprises chemically defined nutrient sources comprising:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium.

Preferably, the initial chemically defined fermentation medium comprises chemically defined nutrient sources comprising:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium; and

optionally one or more selected from the group consisting of

50 μmol to 5 mmol per liter of initial medium iron;

40 μmol to 4 mmol per liter of initial medium copper;

30 μmol to 3 mmol per liter of initial medium manganese, and

40 μmol to 2 mmol per liter of initial medium zinc, and

optionally one or more selected from the group consisting of

1 μmol to 100 μmol per liter of initial medium cobalt;

2 μmol to 200 μmol per liter of initial medium nickel; and

0.3 μmol to 50 μmol per liter of initial medium molybdenum.

In one embodiment, the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, i.e., to the fermentation broth, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions;

wherein the initial chemically defined fermentation medium comprises one or more selected from the group consisting of:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium.

Preferably, in the fermentation process of the present invention, one or more feed solutions comprising one or more trace element ions are added. Preferably, the one or more trace element feeding solutions are added continuously. These trace element ions are selected from the group consisting of iron, copper, manganese, and zinc. Also one or more trace element selected from cobalt, nickel, molybdenum, selenium, and boron can be added. Preferably, the trace element ions iron, copper, manganese, and zinc are added, and optionally one or more selected from cobalt, nickel, and molybdenum are added to the fermentation medium. The trace element ions can be added by one or more feed solutions. The feed solutions can comprise one or more or all trace element ions. Preferably, the trace element ions added via one or more feed solutions to the fermentation broth during cultivation of the cells are iron, copper, manganese, and zinc, and optionally one or more of cobalt, nickel, and molybdenum. Preferably, the one or more trace element ions are added to the fermentation broth during the cultivation of the Bacillus cell by one or more feed solutions comprising one or more trace element ions in an amount selected from the group consisting of at least 50 μmol per liter of initial medium iron, at least 40 μmol per liter of initial medium copper, at least 30 μmol per liter of initial medium manganese, at least 40 μmol per liter of initial medium zinc, and optionally one or more trace element ions in an amount selected from the group consisting of at least 1 μmol per liter of initial medium cobalt, at least 2 μmol per liter of initial medium nickel, and at least 0.3 μmol per liter of initial medium molybdenum. The addition of at least parts of the trace element ions as feed solution to the fermentation broth during cultivation of the cells further increases the titer of the protein of interest. Preferably, at least 50% of the trace element ions are provided in the fermentation process as feed solution, more preferred at least 60%, at least 70%, at least 80%, at least 90%, or 100% of the trace element ions provided in the fermentation process are provided as feed solution to the fermentation process. More preferably, at least 90% of the trace element ions provided in the fermentation process are provided as feed solution to the fermentation process.

For adding the trace element ions one or more from the group consisting of chloride, phosphate, sulphate, nitrate, citrate and acetate salts or trace element hydroxides or combinations of one or more trace element salts and one or more trace element hydroxides can be used.

Thus, in a preferred embodiment the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, and
   wherein the total amount of chemically defined carbon source, preferably glucose, added in the fermentation process is above 200 g of carbon source per liter of initial fermentation medium; and
   wherein at least 0.1 gram magnesium ions per liter of initial fermentation medium is added to the fermentation medium during the cultivation of the Bacillus cell by the one or more feed solutions comprising the magnesium ions; and

wherein one or more trace element ions are added during the cultivation of the Bacillus cell by one or more feed solutions comprising one or more trace element ions in an amount selected from the group consisting of at least 50 μmol per liter of initial medium iron, at least 40 μmol per liter of initial medium copper, at least 30 μmol per liter of initial medium manganese, and at least 40 μmol per liter of initial medium zinc, and in addition optionally one or more selected from the group consisting of at least 1 μmol per liter of initial medium cobalt, at least 2 μmol per liter of initial medium nickel, at least 0.3 μmol per liter of initial medium molybdenum.

Preferably, the trace element ions are added during the cultivation of the Bacillus cell an amount of at least 50 μmol per liter of initial medium iron, at least 40 μmol per liter of initial medium copper, at least 30 μmol per liter of initial medium manganese, and at least 40 μmol per liter of initial medium zinc during the cultivation of the Bacillus cell by one or more feed solutions comprising one or more trace element ions. Preferably, the trace element ions are added during the cultivation of the Bacillus cell an amount of 50 μmol to 5 mmol per liter of initial medium iron, 40 μmol to 4 mmol per liter of initial medium copper, 30 μmol to 3 mmol per liter of initial medium manganese, and 40 μmol to 2 mmol per liter of initial medium zinc during the cultivation of the Bacillus cell by the one or more feed solutions comprising one or more trace element ions. Preferably, the trace element ions are added during the cultivation of the Bacillus cell an amount of at least 50 μmol per liter of initial medium iron, at least 40 μmol per liter of initial medium copper, at least 30 μmol per liter of initial medium manganese, and at least 40 μmol per liter of initial medium zinc, and in addition optionally one or more selected from the group consisting of at least 1 μmol per liter of initial medium cobalt, at least 2 μmol per liter of initial medium nickel, at least 0.3 μmol per liter of initial medium molybdenum during the cultivation of the Bacillus cell by one or more feed solutions comprising one or more trace element ions. Preferably, the trace element ions are added during the cultivation of the Bacillus cell an amount of 50 μmol to 5 mmol per liter of initial medium iron, 40 μmol to 4 mmol per liter of initial medium copper, 30 μmol to 3 mmol per liter of initial medium manganese, and 40 μmol to 2 mmol per liter of initial medium zinc, and in addition optionally one or more selected from the group consisting of 1 μmol to 100 μmol per liter of initial medium cobalt, 2 μmol to 200 μmol per liter of initial medium nickel, 0.3 μmol to 50 μmol per liter of initial medium molybdenum during the cultivation of the Bacillus cell by the one or more feed solutions comprising one or more trace element ions.

Preferably, the trace element ions added to the fermentation medium during the cultivation of the cells by one or more feed solutions comprising the trace element ions are at least 50 μmol per liter of initial medium iron. Preferably, the trace element ions added to the fermentation medium during the cultivation of the cells by one or more feed solutions comprising the trace element ions are 50 μmol to 5 mmol per liter of initial medium iron.

More preferably, the trace element ions added to the fermentation medium during the cultivation of the cells by one or more feed solutions comprising the trace element ions are at least 50 μmol per liter of initial medium iron and at least 40 μmol per liter of initial medium copper. More preferably, the trace element ions added to the fermentation medium during the cultivation of the cells by one or more feed solutions comprising the trace element ions are 50 μmol to 5 mmol per liter of initial medium iron and 40 μmol to 4 mmol per liter of initial medium copper. Even more preferably, the trace element ions are added to the fermentation medium during the cultivation of the cells by one or more feed solutions comprising the trace element ions are at least 50 μmol per liter of initial medium iron, at least 40 μmol per liter of initial medium copper, and at least 30 μmol per liter of initial medium manganese. Even more preferably, the trace element ions are added to the fermentation medium during the cultivation of the cells by one or more feed solutions comprising the trace element ions are 50 μmol to 5 mmol per liter of initial medium iron, 40 μmol to 4 mmol per liter of initial medium copper, and 30 μmol to 3 mmol per liter of initial medium manganese.

More preferably, the trace element ions are added to the fermentation medium during the cultivation of the cells by one or more feed solutions comprising the trace element ions are at least 50 μmol per liter of initial medium iron, at least 40 μmol per liter of initial medium copper, at least 30 μmol per liter of initial medium manganese, and at least 40 μmol per liter of initial medium zinc. More preferably, the trace element ions are added to the fermentation medium during the cultivation of the cells by one or more feed solutions comprising the trace element ions are 50 μmol to 5 mmol per liter of initial medium iron, 40 μmol to 4 mmol per liter of initial medium copper, 30 μmol to 3 mmol per liter of initial medium manganese, and 40 μmol to 2 mmol per liter of initial medium zinc.

More preferably, the trace element ions are added to the fermentation medium in an amount of at least 50 μmol per liter of initial medium iron, at least 40 μmol per liter of initial medium copper, at least 30 μmol per liter of initial medium manganese, and at least 40 μmol per liter of initial medium zinc, and optionally one or more additional trace element ions in an amount selected from the group consisting of at least 1 μmol per liter of initial medium cobalt, at least 2 μmol per liter of initial medium nickel, and at least 0.3 μmol per liter of initial medium molybdenum during the cultivation of the Bacillus cell by one or more feed solutions comprising one or more trace element ions. More preferably, the trace element ions are added to the fermentation medium in an amount of 50 μmol to 5 mmol per liter of initial medium iron, 40 μmol to 4 mmol per liter of initial medium copper, 30 μmol to 3 mmol per liter of initial medium manganese, and 40 μmol to 2 mmol per liter of initial medium zinc, and optionally one or more additional trace element ions in an amount selected from the group consisting of 1 μmol to 100 μmol per liter of initial medium cobalt, 2 μmol to 200 μmol per liter of initial medium nickel, and 0.3 μmol to 50 μmol per liter of initial medium molybdenum during the cultivation of the Bacillus cell by the one or more feed solutions comprising one or more trace element ions.

More preferably, the trace element ions are added to the fermentation medium during the cultivation of the cells by one or more feed solutions comprising the trace element ions in an amount selected from the group consisting of at least 50 μmol per liter of initial medium iron, at least 40 μmol per liter of initial medium copper, at least 30 μmol per liter of initial medium manganese, at least 40 μmol per liter of initial medium zinc, and at least 1 μmol per liter of initial medium cobalt. More preferably, the trace element ions are added to the fermentation medium during the cultivation of the cells by one or more feed solutions comprising the trace element ions in an amount selected from the group consisting of 50 μmol to 5 mmol per liter of initial medium iron, 40 μmol to 4 mmol per liter of initial medium copper, 30 μmol to 3 mmol per liter of initial medium manganese, 40 μmol to 2 mmol per liter of initial medium zinc, and 1 μmol to 100 μmol per liter of initial medium cobalt.

More preferably, the trace element ions are added to the fermentation medium during the cultivation of the cells by one or more feed solutions comprising the trace element ions in an amount selected from the group consisting of at least 50 μmol per liter of initial medium iron, at least 40 μmol per liter of initial medium copper, at least 30 μmol per liter of initial medium manganese, at least 40 μmol per liter of initial medium zinc, at least 1 μmol per liter of initial medium cobalt, and at least 2 μmol per liter of initial medium nickel. More preferably, the trace element ions are added to the fermentation medium during the cultivation of the cells by one or more feed solutions comprising the trace element ions in an amount selected from the group consisting of 50 μmol to 5 mmol per liter of initial medium iron, 40 μmol to 4 mmol per liter of initial medium copper, 30 μmol to 3 mmol per liter of initial medium manganese, 40 μmol to 2 mmol per liter of initial medium zinc, 1 μmol to 100 μmol per liter of initial medium cobalt, and 2 μmol to 200 μmol per liter of initial medium nickel.

Most preferably, the trace element ions are added to the fermentation medium during the cultivation of the cells by one or more feed solutions comprising the trace element ions in an amount selected from the group consisting of at least 50 μmol per liter of initial medium iron, at least 40 μmol per liter of initial medium copper, at least 30 μmol per liter of initial medium manganese, at least 40 μmol per liter of initial medium zinc, at least 1 μmol per liter of initial medium cobalt, at least 2 μmol per liter of initial medium nickel, and at least 0.3 μmol per liter of initial medium molybdenum. Most preferably, the trace element ions are added to the fermentation medium during the cultivation of the cells by one or more feed solutions comprising the trace element ions in an amount selected from the group consisting of 50 μmol to 5 mmol per liter of initial medium iron, 40 μmol to 4 mmol per liter of initial medium copper, 30 μmol to 3 mmol per liter of initial medium manganese, 40 μmol to 2 mmol per liter of initial medium zinc, 1 μmol to 100 μmol per liter of initial medium cobalt, 2 μmol to 200 μmol per liter of initial medium nickel, and 0.3 μmol to 50 μmol per liter of initial medium molybdenum.

Preferably, the trace element ions added to the fermentation medium during the cultivation of the cells by one or more feed solutions comprising the trace element ions further comprise at least 1 μmol per liter of initial medium selenium and/or at least 1 μmol per liter of initial medium boron. Preferably, the trace element ions added to the fermentation medium during the cultivation of the cells by one or more feed solutions comprising the trace element ions further comprise 1 μmol to 200 μmol per liter of initial medium selenium and/or 1 μmol to 200 μmol per liter of initial medium boron.

Preferably, the one or more trace element ions are added to the fermentation broth during the cultivation of the Bacillus cell by one or more feed solutions comprising one or more trace element ions in an amount selected from the group consisting of at least 50 μmol per liter of initial medium iron, at least 40 μmol per liter of initial medium copper, at least 30 μmol per liter of initial medium manganese, and at least 40 μmol per liter of initial medium zinc, and optionally one or more additional trace element ions in an amount selected from the group consisting of at least 1 μmol per liter of initial medium cobalt, at least 2 μmol per liter of initial medium nickel, and at least 0.3 μmol per liter of initial medium molybdenum.

Preferably, the one or more trace element ions are added to the fermentation broth during the cultivation of the Bacillus cell by one or more feed solutions comprising one or more trace element ions in an amount selected from the group consisting of at most 5 mmol per liter of initial medium iron, at most 4 mmol per liter of initial medium copper, at most 3 mmol per liter of initial medium manganese, and at most 2 mmol per liter of initial medium zinc, and optionally one or more additional trace element ions in an amount selected from the group consisting of at most 100 μmol per liter of initial medium cobalt, at most 200 μmol per liter of initial medium nickel, and at most 50 μmol per liter of initial medium molybdenum.

Preferably, the one or more trace element ions are added to the fermentation broth during the cultivation of the Bacillus cell by one or more feed solutions comprising one or more trace element ions in an amount selected from the group consisting of 50 μmol to 5 mmol per liter of initial medium iron, 40 μmol to 4 mmol per liter of initial medium copper, 30 μmol to 3 mmol per liter of initial medium manganese, and 40 μmol to 2 mmol per liter of initial medium zinc, and optionally one or more additional trace element ions in an amount selected from the group consisting of 1 μmol to 100 μmol per liter of initial medium cobalt, 2 μmol to 200 μmol per liter of initial medium nickel, and 0.3 μmol to 50 μmol per liter of initial medium molybdenum.

More, preferably, the one or more trace element ions are added to the fermentation broth during the cultivation of the Bacillus cell by one or more feed solutions comprising one or more trace element ions in an amount selected from the group consisting of at least 250 μmol per liter of initial medium iron, at least 200 μmol per liter of initial medium copper, at least 150 μmol per liter of initial medium manganese, and at least 100 μmol per liter of initial medium zinc, and optionally one or more additional trace element ions in an amount selected from the group consisting of at least 7 μmol per liter of initial medium cobalt, at least 15 μmol per liter of initial medium nickel, and at least 1 μmol per liter of initial medium molybdenum.

More, preferably, the one or more trace element ions are added to the fermentation broth during the cultivation of the Bacillus cell by one or more feed solutions comprising one or more trace element ions in an amount selected from the group consisting of 250 μmol to 5 mmol per liter of initial medium iron, 200 μmol to 4 mmol per liter of initial medium copper, 150 μmol to 3 mmol per liter of initial medium manganese, and 100 μmol to 2 mmol per liter of initial medium zinc, and optionally one or more additional trace element ions in an amount selected from the group consisting of 7 μmol to 100 μmol per liter of initial medium cobalt, 15 μmol to 200 μmol per liter of initial medium nickel, and 1 μmol to 50 μmol per liter of initial medium molybdenum.

Preferably, the one or more trace element ions are added to the fermentation broth during the cultivation of the Bacillus cell by one or more feed solutions comprising one or more trace element ions in an amount selected from the group consisting of 250 μmol to 2 mmol per liter of initial medium iron, 80 μmol to 1.5 mmol per liter of initial medium copper, 150 μmol to 2 mmol per liter of initial medium manganese, and 100 μmol to 1.5 mmol per liter of initial medium zinc, and optionally one or more additional trace element ions in an amount selected from the group consisting of 5 μmol to 70 μmol per liter of initial medium cobalt, 10 μmol to 100 μmol per liter of initial medium nickel, and 1 μmol to 30 μmol per liter of initial medium molybdenum.

Preferably, the one or more trace element ions are added to the fermentation broth during the cultivation of the Bacillus cell by one or more feed solutions comprising one or more trace element ions in an amount selected from the group consisting of 250 μmol to 1 mmol per liter of initial medium iron, 200 μmol to 1 mmol per liter of initial medium copper, 150 μmol to 1 mmol per liter of initial medium manganese, and 100 μmol to 1 mmol per liter of initial medium zinc, and optionally one or more additional trace element ions in an amount selected from the group consisting of 7 μmol to 70 μmol per liter of initial medium cobalt, 15 μmol to 80 μmol per liter of initial medium nickel, and 1 μmol to 20 μmol per liter of initial medium molybdenum.

In one embodiment, at least 70%, at least 80%, at least 90%, or 100% of the carbon, at least 70%, at least 80%, at least 90%, or 100% of the chemically defined trace element ion source and at least 70%, at least 80%, at least 90%, or 100% of the magnesium ions provided in the fermentation process is provided as feed solution in the fermentation process.

In one embodiment, the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, i.e., to the fermentation broth, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions;

wherein one or more chemically defined nutrient sources are added in the fermentation process comprising one or more selected from the group consisting of:

0.1-5 g nitrogen per liter of initial fermentation medium;

1-6 g phosphorus per liter of initial fermentation medium;

0.15-2 g sulfur per liter of initial fermentation medium;

0.4-8 g potassium per liter of initial fermentation medium;

0.1-2 g sodium per liter of initial fermentation medium; and

0.01-3 g calcium per liter of initial fermentation medium; and

wherein also at least parts of the chemically defined nitrogen source, at least parts of the trace element ion source, and at least parts of the sulfur source as described herein are provided by one or more feed solutions during the cultivation of the cells.

In one embodiment, at least 70%, at least 80%, at least 90%, or 100% of the carbon, at least 70%, at least 80%, at least 90%, or 100% of the chemically defined nitrogen source, at least 70%, at least 80%, at least 90%, or 100% of the chemically defined magnesium ion source, at least 70%, at least 80%, at least 90%, or 100% of the chemically defined trace element ion source, and at least 70%, at least 80%, at least 90%, or 100% of the chemically defined sulfur source is provided in the fermentation process is provided as feed solution in the fermentation process.

Preferably, no compound is added during the fermentation process in an amount that the protein of interest precipitates in the form of crystals and/or amorphous precipitates from solution. Preferably, no sulfate salts, preferably not ammonium sulfate, are added during cultivation of the cells in an amount that the protein of interest precipitates from solution.

Thus, in a preferred embodiment the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, and
   wherein the total amount of chemically defined carbon source, preferably glucose, added in the fermentation process is above 200 g of carbon source per liter of initial fermentation medium; and
   wherein at least 0.1 gram magnesium ions per liter of initial fermentation medium is added to the fermentation medium during the cultivation of the Bacillus cell by the one or more feed solutions comprising the magnesium ions; and
   wherein the fermentation process does not comprise a step of precipitating the protein of interest by adding a compound to the fermentation medium during cultivation of the cells in an amount that leads to precipitation of the protein of interest.

Preferably, the fermentation medium prior inoculation of the cells comprises one or more compounds selected from the group consisting of a chemically defined nitrogen source, a chemically defined calcium source, a chemically defined potassium source, a chemically defined phosphorus source, a chemically defined magnesium source, a chemically defined sulfur source, a chemically defined sodium source, and a chemically defined chelating agent in water. Preferably, the fermentation medium prior inoculation of the cells comprises a chemically defined nitrogen source, a chemically defined calcium source, a chemically defined potassium source, a chemically defined phosphorus source, a chemically defined magnesium source, a chemically defined sulfur source, a chemically defined sodium source, and a chemically defined chelating agent in water. More preferably, the fermentation medium prior inoculation of the cells comprises a calcium salt, KH2PO4, MgSO4, citric acid, and water. Further preferred, the fermentation medium prior inoculation of the cells comprises as medium components in water only a chemically defined nitrogen source, chemically defined calcium source, a chemically defined potassium source, a chemically defined phosphorus source, a chemically defined magnesium source, a chemically defined sulfur source, a chemically defined sodium source, one or more chemically defined trace element ion sources, and optionally a chelating agent. Even more preferred, the fermentation medium prior inoculation of the cells comprises as medium components in water only ammonia, a calcium salt, a potassium salt, a salt comprising phosphorus, a salt comprising sulfur, sodium hydroxide, a magnesium salt, and one or more trace element ion salts, and optionally a chelating agent. Most preferred, the fermentation medium prior inoculation of the cells comprises as medium components in water only ammonia, a calcium salt, a potassium salt, a salt comprising phosphate, a salt comprising sulphate, sodium hydroxide, a magnesium salt, one or more trace element ion salts, preferably the trace elements being selected from the group consisting of Fe, Cu, Mn, and Zn, and optionally in addition one or more trace elements selected from Co, Ni, and Mo, preferably all of Fe, Cu, Mn, and Zn, and preferably in addition one or more trace elements selected from Co, Ni, and Mo, and optionally a chelating agent, which is preferably citrate.

Preferably the amount of chemically defined carbon source, preferably glucose, in the initial fermentation medium prior inoculation of the cells is below 50%, below 40%, below 30%, preferably below 20%, or more preferably at most 10% of the amount of chemically defined carbon source provided to the fermentation medium in the fermentation process.

Preferably the amount of magnesium ions in the initial fermentation medium prior inoculation of the cells is below 50%, below 40%, below 30%, preferably below 20%, or more preferably at most 10% of the amount of magnesium ions provided to the fermentation medium in the fermentation process.

Preferably the amount of trace element ions in the initial fermentation medium prior inoculation of the cells is below 50%, below 40%, below 30%, preferably below 20%, or more preferably at most 10% of the amount of trace element ions provided to the fermentation medium in the fermentation process.

Preferably, the pH of the fermentation broth during cultivation of the Bacillus cells is adjusted to at or above pH 6.0, pH 6.5, pH 7.0, pH 7.2, pH 7.4, or pH 7.6. Preferably, the pH of the fermentation broth during cultivation of the Bacillus cells is adjusted to pH 6.6 to 9, preferably to pH 6.6 to 8.5, more preferably to pH 7.0 to 8.5, most preferably to pH 7.2 to pH 8.0. Preferably, the pH of the fermentation broth during cultivation is adjusted with ammonia and/or sodium hydroxide, preferably with sodium hydroxide and ammonia. In a preferred embodiment of the present invention, the chemically defined nitrogen source is ammonia and is added in the fermentation process only in an amount necessary for pH adjustment. This allows for a complete conversion of the chemically defined nitrogen source to the protein of interest and biomass generation without unnecessary formation of salts. In this embodiment a separate chemically defined nitrogen source feed can be omitted. In case sodium hydroxide is used for pH adjustment also no additional sodium source needs to be fed.

In one embodiment, at least 50% of the chemically defined nitrogen source is provided in the fermentation process as feed solution, more preferred at least 60%, at least 70%, at least 80%, at least 90%, or 100% of the chemically defined nitrogen source is provided as feed solution in the fermentation process. Preferably the amount of the chemically defined nitrogen source in the initial fermentation medium prior inoculation of the cells is below 50%, preferably below 40%, below 30%, below 20%, or below 10% of the amount of chemically defined nitrogen source provided to the fermentation medium in the fermentation process.

The total amount of chemically defined nitrogen source added to the chemically defined medium during the fermentation process may vary from 0.5 to 50 g nitrogen (N) per liter of initial fermentation medium, preferably from 1 to 25 g N per liter of initial fermentation medium, more preferably from 10 to 25 g N per liter of initial fermentation medium, wherein N is expressed as Kjeldahl nitrogen. Preferably, the ratio between chemically defined carbon and chemically defined nitrogen source added during a fermentation process may vary, whereby one determinant for an optimal ratio between chemically defined carbon and chemically defined nitrogen source is the elemental composition of the protein of interest to be formed.

Preferably, the fermentation process of the present invention is not conducted under nitrogen limitation. More preferably, the fermentation process of the present invention is not conducted under ammonia limitation.

Thus, in a preferred embodiment the present invention refers to a fermentation process for cultivating a Bacillus cell in a chemically defined fermentation medium comprising the steps of
(a) providing a chemically defined fermentation medium,
(b) inoculating this initial fermentation medium with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium comprising the cells, and
   wherein the total amount of chemically defined carbon source, preferably glucose, added in the fermentation process is above 200 g of carbon source per liter of initial fermentation medium; and
   wherein at least 0.1 gram magnesium ions per liter of initial fermentation medium is added to the fermentation medium during the cultivation of the Bacillus cell by the one or more feed solutions comprising the magnesium ions; and
   wherein the one or more trace element ions are added to the fermentation medium during the cultivation of the Bacillus cell by one or more feed solutions comprising one or more trace element ions in an amount selected from the group consisting of at least 50 μmol per liter of initial medium iron, at least 40 μmol per liter of initial medium copper, at least 30 μmol per liter of initial medium manganese, and at least 40 μmol per liter of initial medium zinc, and optionally one or more additional trace element ions in an amount selected from the group consisting of at least 1 μmol per liter of initial medium cobalt, at least 2 μmol per liter of initial medium nickel, at least 0.3 μmol per liter of initial medium molybdenum; and wherein at least 0.5 g N from the chemically defined nitrogen source, preferably ammonia, per liter of initial fermentation medium is added to the fermentation medium during the cultivation of the Bacillus cell by the one or more feed solutions comprising the chemically defined nitrogen source.

Preferably, the temperature of the fermentation broth during cultivation is 25° C. to 45° C., preferably, 27° C. to 40° C., more preferably, 27° C. to 37° C.

Preferably, oxygen is added to the fermentation medium during cultivation, preferably by agitation and gassing, preferably with 0-3 bar air or oxygen.

Preferably, the fermentation time is 1-200 hours, preferably, 1-120 hours, more preferably 10-90 h, even more preferably, 20-70 h.

Host Cell

The fermentation process of the present invention is for producing a protein of interest in a Bacillus cell.

The Bacillus cell is preferably a Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus licheniformis, Bacillus megaterium, Bacillus pumilus, Bacillus stearothermophilus, Bacillus subtilis, Bacillus thuringiensis and Bacillus velezensis. Preferably, the Bacillus is a Bacillus cell of Bacillus subtilis, Bacillus pumilus, Bacillus licheniformis, or Bacillus lentus. Preferably, the Bacillus is a Bacillus licheniformis, a Bacillus subtilis or a Bacillus pumilus. Most preferred is a Bacillus licheniformis, preferably, Bacillus licheniformis ATCC53926.

The Bacillus cell can comprise the gene encoding the protein of interest (i.e., gene of interest) endogenously or the gene of interest can be heterologous to the Bacillus cell. Preferably, the gene encoding the protein of interest is heterologous to the host cell.

The nucleic acid construct comprising the gene encoding the protein of interest comprises one or more inducer-independent promoter sequences that directs the expression of the gene of interest in the Bacillus cell and further comprises a transcription and translation start and terminator.

The inducer-independent promoter sequence can be native or heterologous to the host cell.

Preferably, the inducer-independent promoter sequence is a constitutive promoter sequence, preferably a sigma A dependent promoter sequence, or a promoter sequence that is regulated by factors other than an inducer molecule that is added to the fermentation medium.

Preferably, the inducer-independent promoter sequence is selected from the group consisting of constitutive, sigma A dependent promoter sequences (preferably as described in Helmann, J. D. 1995. Compilation and analysis of Bacillus subtilis sigma A-dependent promoter sequences: evidence for extended contact between RNA polymerase and upstream promoter DNA. Nucleic Acids Res. 23(13), 2351-2360), preferably, the promoter sequence of Pveg, PlepA, PserA, PymdA, or Pfba, and derivatives thereof with different strength of gene expression (preferably as described in Guiziou, S., Sauveplane, V., Chang, H. J., Clerte, C., Declerck, N., Jules, M., and Bonnet, 2016. J. A part toolbox to tune genetic expression in Bacillus subtilis. Nucleic Acids Res. 44(15), 7495-7508), and combinations thereof, and active fragments or variants thereof.

Alternatively, the inducer-independent promoter sequence that is regulated by factors other than an inducer molecule that is added to the fermentation medium is selected from the group consisting of the promoter sequences of the aprE promoter, amyQ promoter from Bacillus amyloliquefaciens, amyL promoter and variants thereof from Bacillus licheniformis (preferably as described in U.S. Pat. No. 5,698,415), bacteriophage SPO1 promoter, preferably the promoter P4, P5, or P15 (preferably as described in WO15118126 or in Stewart, C. R., Gaslightwala, I., Hinata, K., Krolikowski, K. A., Needleman, D. S., Peng, A. S., Peterman, M. A., Tobias, A., and Wei, P. 1998, Genes and regulatory sites of the “host-takeover module” in the terminal redundancy of Bacillus subtilis bacteriophage SPO1. Virology 246(2), 329-340), cryIIIA promoter from Bacillus thuringiensis (preferably as described in WO9425612 or in Agaisse, H. and Lereclus, D. 1994. Structural and functional analysis of the promoter region involved in full expression of the cryIIIA toxin gene of Bacillus thuringiensis. Mol. Microbiol. 13(1). 97-107), and combinations thereof, and active fragments or variants thereof.

Preferably, the promoter sequences can be combined with 5′-UTR sequences native or heterologous to the host cell, as described herein.

Preferably, the promoter sequence selected from the group consisting of an veg promoter, lepA promoter, serA promoter, ymdA promoter, fba promoter, aprE promoter, amyQ promoter, amyL promoter, bacteriophage SPO1 promoter, cryIIIA promoter, combinations thereof, and active fragments or variants thereof. More preferably, the inducer-independent promoter sequence is selected from the group consisting of aprE promoter, amyL promoter, veg promoter, bacteriophage SPO1 promoter, cryIIIA promoter and combinations thereof, or active fragments or variants thereof, preferably an aprE promoter sequence.

In a further preferred embodiment, the inducer-independent promoter sequence is selected from the group consisting of aprE promoter, SPO1 promoter, preferably P4, P5, or P15 (preferably as described in WO15118126), tandem promoter comprising the promoter sequences amyl and amyQ (preferably as described in WO9943835), and triple promoter comprising the promoter sequences amyL, amyQ, and cryIIIa (preferably as described in WO2005098016).

Preferably, the inducer-independent promoter sequence is an aprE promoter sequence.

In a preferred embodiment, the expression of the gene of interest in the Bacillus cell is under the control of the native promoter from the gene encoding the Bacillus subtilisin Carlsberg protease, also referred to as aprE promoter, or an active fragment or an active variant thereof.

The native promoter from the gene encoding the Bacillus subtilisin Carlsberg protease, also referred to as aprE promoter, is well described in the art. The aprE gene is transcribed by sigma factor A (sigA) and its expression is highly controlled by several regulators—DegU acting as activator of aprE expression, whereas AbrB, ScoC (hpr) and SinR are repressors of aprE expression (Ferrari, E., D. J. Henner, M. Perego, and J. A. Hoch. 1988. Transcription of Bacillus subtilis subtilisin and expression of subtilisin in sporulation mutants. J Bacteriol 170: 289-295; Henner, D. J., E. Ferrari, M. Perego, and J. A. Hoch. 1988. Location of the targets of the hpr-97, sacU32(Hy), and sacQ36(Hy) mutations in upstream regions of the subtilisin promoter. J. Bacteriol. 170: 296-300; Park, S. S., S. L. Wong, L. F. Wang, and R. H. Doi. 1989. Bacillus subtilis subtilisin gene (aprE) is expressed from a sigma A (sigma 43) promoter in vitro and in vivo. J Bacteriol 171: 2657-2665; Gaur, N. K., J. Oppenheim, and I. Smith. 1991. The Bacillus subtilis sin gene, a regulator of alternate developmental processes, codes for a DNA-binding protein. J Bacteriol 173: 678-686; Kallio, P. T., J. E. Fagelson, J. A. Hoch, and M. A. Strauch. 1991. The transition state regulator Hpr of Bacillus subtilis is a DNA-binding protein. Journal of Biological Chemistry 266: 13411-13417). The core promoter region comprising the sigma factor A binding sites −35 and −10 have been mapped to the region nt-1-nt-45 relative to the transcriptional start site (Park, S. S., S. L. Wong, L. F. Wang, and R. H. Doi. 1989. Bacillus subtilis subtilisin gene (aprE) is expressed from a sigma A (sigma 43) promoter in vitro and in vivo. J Bacteriol 171: 2657-2665). WO0151643 describes the increase of expression by mutating the −35 site of the wild type aprE promoter from TACTAA to the canonical TTGACA −35 site motif (Helmann, J. D. 1995. Compilation and analysis of Bacillus subtilis sigma A-dependent promoter sequences: evidence for extended contact between RNA polymerase and upstream promoter DNA. Nucleic Acids Res. 23: 2351-2360).

The transcriptional start site (TSS) is located at nt-58 relative to the start GTG of the aprE gene. The 5′UTR comprises the ribosome binding site (Shine Dalgarno) and a sequence within nt-58-nt-33 relative to the start GTG forming a very stable stem-loop structure of the 5′-end of the mRNA being responsible for high mRNA transcript stability of up to 25 min (Hambraeus et al., 2000; Hambraeus et al., 2002). The region of nt-141-nt-161 relative to the transcriptional start site has be shown to be responsible for full induction in a DegU (SacU) and DegQ (SacQ) dependent manner, whereas regions 5′ of nt-200 up to nt-600 are negatively regulated by ScoC (Hpr) (Henner, D. J., E. Ferrari, M. Perego, and J. A. Hoch. 1988. Location of the targets of the hpr97, sacU32(Hy), and sacQ36(Hy) mutations in upstream regions of the subtilisin promoter. J. Bacteriol. 170: 296-300). The ScoC (hpr) binding sites within the Bacillus subtilis aprE promoter region have been more precisely mapped revealing additional binding sites within the abovementioned core promoter region (Kallio, P. T., J. E. Fagelson, J. A. Hoch, and M. A. Strauch. 1991. The transition state regulator Hpr of Bacillus subtilis is a DNA-binding protein. Journal of Biological Chemistry 266: 13411-13417). The binding site of the repressing transition state regulator ArbB has been mapped to nt-58-+nt 15 relative to the transcriptional start site (Strauch, M. A., G. B. Spiegelman, M. Perego, W. C. Johnson, D. Burbulys, and J. A. Hoch. 1989. The transition state transcription regulator abrB of Bacillus subtilis is a DNA binding protein. EMBO J 8: 1615-1621). The bindging sites of the repressor SinR have been mapped to nt-233-nt-268 relative to the transcriptional start site (Gaur, N. K., J. Oppenheim, and I. Smith. 1991. The Bacillus subtilis sin gene, a regulator of alternate developmental processes, codes for a DNA-binding protein. J Bacteriol 173: 678-686).

Jakobs et al (Jacobs, M., M. Eliasson, M.Uhl+®n, and J. I. Flock. 1985. Cloning, sequencing and expression of subtilisin Carlsberg from Bacillus licheniformis. Nucleic Acids Res 13: 8913-8926; Jacobs, M. F. 1995. Expression of the subtilisin Carlsberg-encoding gene in Bacillus licheniformis and Bacillus subtilis. Gene 152: 69-74) discloses the sequence of the aprE (subC) gene and its 5′ region of the Bacillus licheniformis NCIB6816 strain (GenBank accession No. X03341). The regulation of the expression of the subtilisin Carlsberg aprE (subC) gene and the DNA sequences involved are described. The transcriptional start site (TSS) is located at nt-73 and accordingly the 5′ UTR comprising nt-73-nt-1 relative to the Start ATG. The ribosome binding site (Shine Dalgarno) is located at position nt-16-nt-9. The recognition sequence −10-site (TATAAT-box) of the sigma factor A is highly conserved and located at nt-84-nt-79 whereas the −35 site (TACCAT) located 17 nt upstream of the −10 site is less conserved compared to standard sigma factor A dependent promoters in Bacillus (Helmann, 1995). Promoter truncations from the 5′ end comprising nt-122-nt-1 and nt-181-nt-1 (mutant 771 and mutant 770, respectively, as described in Jacobs et al., 1995) show 20-40 fold reduced subtilisin Carlsberg protease expression activities compared to expression with promoter fragment nt-225-nt-1 (mutant 769, as described in Jacobs et al., 1995) in Bacillus subtilis strains with elevated regulators DegU (degU32H) or DegQ (degQ36H). Therefore, the binding sites of the regulator degU stimulating subtilisin Carlsberg expression lie within the region comprising nt-225-nt-182.

WO9102792 discloses the functionality of the promoter of the ATCC 53926 alkaline protease gene for the large-scale production of subtilisin Carlsberg-type protease in Bacillus licheniformis ATCC 53926. The subtilisin Carlsberg is produced in a fermentation process using complex media components as nitrogen and carbon sources.

In particular, WO9102792 describes the 5′ region of the subtilisin Carlsberg protease encoding aprE gene of Bacillus licheniformis ATCC 53926 (FIG. 27) comprising the functional aprE gene promoter and the 5′UTR comprising the ribosome binding site (Shine Dalgarno sequence). Moreover, the truncated fragment thereof starting with the Aval restriction endonuclease site comprises the functional aprE gene promoter and the 5′UTR comprising the ribosome binding site (Shine Dalgarno sequence) as exemplified by expression of subtilisin Carlsberg fusion protein consisting of the signal peptide of the aprE gene from Bacillus licheniformis ATCC 53926 and the propeptide sequence and mature sequence of the Bacillus lentus DSM5383 alkaline protease gene.

In a preferred embodiment, the expression of the gene of interest in the Bacillus cell is under the control of the native promoter from the gene encoding the Bacillus subtilisin Carlsberg protease, also referred to as aprE promoter, which are selected from the group of promoters with an HMM-score above 50 or an active fragment or variant thereof.

Preferably, the aprE promoter is selected from the group of aprE promoters from Bacillus amylo liquefaciens, Bacillus clausii, Bacillus haloduans, Bacillus lentus, Bacillus licheniformis, Bacillus pumilus, Bacillus subtilis, or Bacillus velezensis. Preferably, the aprE promoter is from Bacillus licheniformis, Bacillus pumilus, and Bacillus subtilis. Most preferably, the aprE promoter is from Bacillus licheniformis.

More preferably, the aprE promoter is the promoter of the gene coding for the subtilisin Carlsberg protease or a functional fragment of the aprE promoter sequence or a functional variant of the aprE promoter sequence of the gene coding for the subtilisin Carlsberg protease, wherein the subtilisin Carlsberg protease has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 2, SEQ ID NO: 4, or SEQ ID NO: 6.

Preferably, the aprE promoter comprises the sigma factor A core promoter, preferably binding motifs −35 and −10.

Preferably, the aprE promoter comprises one or more of the binding motifs of regulatory factors selected from the group consisting of degU (sacU), ScoC (hpr), SinR and AbrB. Most preferably, the aprE promoter comprises one or more binding motifs of the regulatory factor degU.

Preferably, the aprE promoter comprises the sigma factor A core promoter, preferably binding motifs −35 and −10, and the binding region for the DegU regulator.

In more preferred embodiment the aprE promoter are selected but not limited to the group of promoters with an HMM-score above 50 comprising the sigma factor A core promoter, preferably binding motifs −35 and −10, and preferably the binding region for the DegU regulator.

Preferably, the aprE promoter described herein and used in the methods of the present invention is in one embodiment an aprE promoter having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 8, SEQ ID NO: 10, SEQ ID NO: 12, or SEQ ID NO: 13.

Preferably, the aprE promoter described herein and used in the methods of the present invention is in one embodiment an aprE promoter having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 8, SEQ ID NO: 10, or SEQ ID NO: 12.

Preferably, the aprE promoter described herein and used in the methods of the present invention is in one embodiment an aprE promoter having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 8, SEQ ID NO: 10, or SEQ ID NO: 12, and wherein the aprE promoter comprises the sigma factor A core promoter, preferably binding motifs −35 and −10, and preferably the binding region for the DegU regulator.

Preferably, the aprE promoter described herein and used in the methods of the present invention is in one embodiment an aprE promoter having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 8, SEQ ID NO: 10, or SEQ ID NO: 12, or an active fragment thereof, and wherein the aprE promoter comprises the sigma factor A core promoter, preferably binding motifs −35 and −10, and the binding region for the DegU regulator.

More preferably, the aprE promoter has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 12.

Preferably, the aprE promoter described herein and used in the methods of the present invention is in one embodiment an aprE promoter having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 8, SEQ ID NO: 10, or SEQ ID NO: 12, or an active fragment, wherein the active fragment is selected from a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 13.

Most preferably, the aprE promoter has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 13.

Most preferably, the aprE promoter has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 13 and wherein the aprE promoter comprises the sigma factor A core promoter, preferably binding motifs −35 and −10, and preferably the binding region for the DegU regulator.

Preferably, the aprE promoter is a variant of the aprE promoter sequences shown in SEQ ID NO: 8, 10, 12, or 13. Preferably, the variant of the aprE promoter sequence of SEQ ID NO: 8, 10, 12, or 13 comprises a substitution, deletion, and/or insertion at one or more positions and wherein the variant of the promoter sequence has promoter activity. In one embodiment, the variant of the aprE promoter of SEQ ID NO: 8, 10, 12, or 13 comprising a substitution at one or more positions and having promoter activity comprises up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, or up to 20 substitutions.

In one embodiment, the nucleic acid construct and/or the expression vector comprising the gene of interest comprises in addition to the promoter sequence one or more further control sequences. Preferably, such control sequences enable translation of the gene's mRNA. Such control sequences can be native or heterologous to the host cell. Such control sequences include, but are not limited to 5′-UTR (also called leader sequence), ribosomal binding site (RBS, shine dalgarno sequence), and 3′-UTR. Preferably, the nucleic acid construct and/or the expression vector comprises a 5′-UTR and a RBS. Preferably, the 5′-UTR is selected from the control sequence of a gene selected from the group consisting of aprE, grpE, ctoG, SP82, gsiB, cryIIa and ribG gene.

The desired protein may be secreted (into the liquid fraction of the fermentation broth) or may remain inside the Bacillus cells. Preferably, the fermentation product is secreted by the Bacillus cell into the fermentation broth. Secretion of the protein of interest into the fermentation medium allows for a facilitated separation of the protein of interest from the fermentation medium. For secretion of the protein of interest into the fermentation medium the nucleic acid construct comprises a polynucleotide encoding for a signal peptide that directs secretion of the protein of interest into the fermentation medium. Various signal peptides are known in the art. Preferred signal peptides are selected from the group consisting of the signal peptide of the AprE protein from Bacillus subtilis or the signal peptide from the YvcE protein from Bacillus subitilis.

In particular suitable for secreting amylases from Bacillus cells into the fermentation medium are the signal peptide of the AprE protein from Bacillus subtilis or the signal peptide from the YvcE protein from Bacillus subtilis. As the YvcE signal peptide is suitable for secreting a wide variety of different amylases this signal peptide can be used, preferably in conjunction with the fermentation process described herein, for expressing a variety of amylases and analyzing the amylases regarding their properties, e.g., amylolytic activity or stability.

In one embodiment, the expression vector comprising the gene of interest is located outside the chromosomal DNA of the Bacillus host cell. In another embodiment, the expression vector is integrated into the chromosomal DNA of the Bacillus cell in one or more copies. The expression vector can be linear or circular. In one embodiment, the expression vector is a viral vector or a plasmid.

For autonomous replication, the expression vector may further comprise an origin of replication enabling the vector to replicate autonomously in the host cell in question. Bacterial origins of replication include but are not limited to the origins of replication of plasmids pUB110, pC194, pTB19, pAMß1, and pTA1060 permitting replication in Bacillus (Janniere, L., Bruand, C., and Ehrlich, S. D. (1990). Structurally stable Bacillus subtilis cloning vectors. Gene 87, 53-6; Ehrlich, S. D., Bruand, C., Sozhamannan, S., Dabert, P., Gros, M. F., Janniere, L., and Gruss, A. (1991). Plasmid replication and structural stability in Bacillus subtilis. Res. Microbiol. 142, 869-873), and pE194 (Dempsey, L. A. and Dubnau, D. A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436).

In one embodiment, the expression vector contains one or more selectable markers that permit easy selection of transformed cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO91/09129, where the selectable marker is on a separate vector.

Protein of Interest

The present invention refers to a method of producing a protein of interest comprising the use of the fermentation process as described herein. Thus, the present invention refers to a method of producing a protein of interest comprising the fermentation process described herein in further details comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation broth, and
   wherein the total amount of chemically defined carbon source added in the fermentation process is above 200 g of carbon source per liter of initial fermentation medium, and
   wherein at least 0.1 gram magnesium ions per liter of initial fermentation medium is added to the fermentation medium during the cultivation of the Bacillus cell by the one or more feed solutions comprising the magnesium ions.

Preferably, the protein of interest is expressed in an amount of at least 3 g protein (dry matter)/kg fermentation broth, preferably in an amount of at least 5 g protein (dry matter)/kg fermentation broth, preferably in an amount of at least 10 g protein (dry matter)/kg fermentation broth, preferably in an amount of at least 15 g protein (dry matter)/kg fermentation broth, preferably in an amount of at least 20 g protein (dry matter)/kg fermentation broth.

As the fermentation process of the present invention is suitable to provide high titers of the protein of interest, in one embodiment, the present invention refers to a method for increasing the titer of a protein of interest comprising the fermentation process as described herein. Preferably, the fermentation process provides a titer of at least 5 g/l of protein of interest. More preferably, the fermentation process provides a titer of at least 10 g/l of protein of interest. Even more preferably, the fermentation process provides a titer of at least 15 g/l of protein of interest.

Preferably, the protein of interest is an enzyme. In a particular embodiment, the enzyme is classified as an oxidoreductase (EC 1), a transferase (EC 2), a hydrolase (EC 3), a lyase (EC 4), an isomerase (EC 5), or a ligase (EC 6) (EC-numbering according to Enzyme Nomenclature, Recommendations (1992) of the Nomenclature Committee of the International Union of Biochemistry and Molecular Biology including its supplements published 1993-1999). In a preferred embodiment, the protein of interest is an enzyme suitable to be used in detergents.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, glucoamylase, nuclease, and cellulase, preferably, amylase or protease, preferably, a protease. Most preferred is a serine protease (EC 3.4.21), preferably a subtilisin protease.

In a particular preferred embodiment, the following proteins of interest are preferred:

Protease

Enzymes having proteolytic activity are called “proteases” or “peptidases”. Proteases are active proteins exerting “protease activity” or “proteolytic activity”.

Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyldipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endopeptidases of unknown catalytic mechanism (EC 3.4.99).

Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect® Prime, Purafect MAO, Purafect Ox®, Purafect OxP®, Puramax®, Properase®, FN2®, FN3®, FN4®, Excellase®, Eraser®, Ultimase®, Opticlean®, Effectenz®, Preferenz® and Optimase® (Danisco/DuPont), Axapem™ (Gist-Brocases N.V.), Bacillus lentus Alkaline Protease, and KAP (Bacillus alkalophilus subtilisin) from Kao.

At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”.

A sub-group of the serine proteases tentatively designated subtilases has been proposed by Siezen et al. (1991), Protein Eng. 4:719-737 and Siezen et al. (1997), Protein Science 6:501-523. They are defined by homology analysis of more than 170 amino acid sequences of serine proteases previously referred to as subtilisin-like proteases. A subtilisin was previously often defined as a serine protease produced by Gram-positive bacteria or fungi, and according to Siezen et al. now is a subgroup of the subtilases. A wide variety of subtilases have been identified, and the amino acid sequence of a number of subtilases has been determined. For a more detailed description of such subtilases and their amino acid sequences reference is made to Siezen et al. (1997), Protein Science 6:501-523.

The subtilases may be divided into 6 sub-divisions, i.e. the subtilisin family, thermitase family, the proteinase K family, the lantibiotic peptidase family, the kexin family and the pyrolysin family.

A subgroup of the subtilases are the subtilisins which are serine proteases from the family S8 as defined by the MEROPS database (http://merops.sanger.ac.uk). Peptidase family S8 contains the serine endopeptidase subtilisin and its homologues.

Prominent members of family S8, subfamily A are:

| name | MEROPS Family S8, Subfamily A |
|---|---|
| Subtilisin Carlsberg | S08.001 |
| Subtilisin lentus | S08.003 |
| Thermitase | S08.007 |
| Subtilisin BPN′ | S08.034 |
| Subtilisin DY | S08.037 |
| Alkaline peptidase | S08.038 |
| Subtilisin ALP 1 | S08.045 |
| Subtilisin sendai | S08.098 |
| Alkaline elastase YaB | S08.157 |

Parent proteases of the subtilisin type (EC 3.4.21.62) and variants may be bacterial proteases. Said bacterial protease may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma protease. A review of this family is provided, for example, in Subtilases: Subtilisin-like Proteases” by R. Siezen, pages 75-95 in “Subtilisin enzymes”, edited by R. Bott and C. Betzel, New York, 1996.

At least one protease may be selected from the following: subtilisin from Bacillus amyloliquefaciens BPN′ (described by Vasantha et al. (1984) J. Bacteriol. Volume 159, p. 811-819 and JA Wells et al. (1983) in Nucleic Acids Research, Volume 11, p. 7911-7925); subtilisin from Bacillus licheniformis (subtilisin Carlsberg; disclosed in EL Smith et al. (1968) in J. Biol Chem, Volume 243, pp. 2184-2191, and Jacobs et al. (1985) in Nucl. Acids Res, Vol 13, p. 8913-8926); subtilisin PB92 (original sequence of the alkaline protease PB92 is described in EP 283075 A2); subtilisin 147 and/or 309 (Esperase®, Savinase®, respectively) as disclosed in WO 89/06279; subtilisin from Bacillus lentus as disclosed in WO 91/02792, such as from Bacillus lentus DSM 5483 or the variants of Bacillus lentus DSM 5483 as described in WO 95/23221; subtilisin from Bacillus alcalophilus (DSM 11233) disclosed in DE 10064983; subtilisin from Bacillus gibsonii (DSM 14391) as disclosed in WO 2003/054184; subtilisin from Bacillus sp. (DSM 14390) disclosed in WO 2003/056017; subtilisin from Bacillus sp. (DSM 14392) disclosed in WO 2003/055974; subtilisin from Bacillus gibsonii (DSM 14393) disclosed in WO 2003/054184; subtilisin having SEQ ID NO: 4 as described in WO 2005/063974; subtilisin having SEQ ID NO: 4 as described in WO 2005/103244; subtilisin having SEQ ID NO: 7 as described in WO 2005/103244; and subtilisin having SEQ ID NO: 2 as described in application DE 102005028295.4.

At least one subtilisin may be subtilisin 309 (which might be called Savinase® herein) as disclosed as sequence a) in Table I of WO 89/06279 or a variant which is at least 80% identical thereto and has proteolytic activity.

Proteases are known as comprising the variants described in: WO 92/19729, WO 95/23221, WO 96/34946, WO 98/20115, WO 98/20116, WO 99/11768, WO 01/44452, WO 02/088340, WO 03/006602, WO 2004/03186, WO 2004/041979, WO 2007/006305, WO 2011/036263, WO 2011/036264, and WO 2011/072099. Suitable examples comprise especially protease variants of subtilisin protease derived from SEQ ID NO:22 as described in EP 1921147 (with amino acid substitutions in one or more of the following positions: 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and 274 which have proteolytic activity. In addition, a subtilisin protease is not mutated at positions Asp32, His64 and Ser221.

At least one subtilisin may have SEQ ID NO:22 as described in EP 1921147, or is a variant thereof which is at least 80%, at least 90%, at least 95% or at least 98% identical SEQ ID NO:22 as described in EP 1921147 and has proteolytic activity. In one embodiment, a subtilisin is at least 80%, at least 90%, at least 95% or at least 98% identical to SEQ ID NO:22 as described in EP 1921147 and is characterized by having amino acid glutamic acid (E), or aspartic acid (D), or asparagine (N), or glutamine (Q), or alanine (A), or glycine (G), or serine (S) at position 101 (according to BPN′ numbering) and has proteolytic activity. In one embodiment, subtilisin is at least 80%, at least 90%, at least 95% or at least 98% identical to SEQ ID NO:22 as described in EP 1921147 and is characterized by having amino acid glutamic acid (E) or aspartic acid (D), preferably glutamic acid (E), at position 101 (according to BPN′ numbering) and has proteolytic activity. Such a subtilisin variant may comprise an amino acid substitution at position 101, such as R101E or R101D, alone or in combination with one or more substitutions at positions 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and/or 274 (according to BPN′ numbering) and has proteolytic activity. In a preferred embodiment, the subtilisin protease is identical to SEQ ID NO:22 as described in EP 1921147 except that the protease is characterized by having amino acid glutamic acid (E) at position 101 (according to BPN′ numbering). In one embodiment, said protease comprises one or more further substitutions: (a) threonine at position 3 (3T), (b) isoleucine at position 4 (4I), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) isoleucine at position 205 (2051), (h) aspartic acid, glutamic acid or glycine at position 217 (217D, 217E or 217G), (i) combinations of two or more amino acids according to (a) to (h).

A suitable subtilisin may be at least 80% identical to SEQ ID NO:22 as described in EP 1921147 and is characterized by comprising one amino acid (according to (a)-(h)) or combinations according to (i) together with the amino acid 101E, 101D, 101N, 101Q, 101A, 101G, or 101S (according to BPN′ numbering) and has proteolytic activity.

In one embodiment, a subtilisin is at least 80% identical to SEQ ID NO:22 as described in EP 1921147 and is characterized by comprising the mutation (according to BPN′ numbering) R101E, or S3T+V4I+V2051, or S3T+V4I+R101E+V2051 or S3T+V4I+V199M+V2051+L217D, and has proteolytic activity. If secretion of these proteases into the fermentation medium is desired the use of the signal peptide of the AprE protein from Bacillus subtilis is preferred.

In another embodiment, the subtilisin comprises an amino acid sequence having at least 80% identity to SEQ ID NO:22 as described in EP 1921147 and being further characterized by comprising S3T+V4I+S9R+A15T+V68A+D99S+R101S+A103S+1104V+N218D (according to the BPN′ numbering) and has proteolytic activity.

A subtilisin may have an amino acid sequence being at least 80% identical to SEQ ID NO:22 as described in EP 1921147 and being further characterized by comprising R101E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T,D and L262N,Q,D (as described in WO 2016/096711 and according to the BPN′ numbering), and has proteolytic activity.

Proteases according to the invention have proteolytic activity. The methods for determining proteolytic activity are well-known in the literature (see e.g. Gupta et al. (2002), Appl. Microbiol. Biotechnol. 60: 381-395). Proteolytic activity may be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of yellow color of free pNA which can be quantified by measuring OD405.

Amylase

Alpha-amylase (E.C. 3.2.1.1) enzymes may perform endohydrolysis of (1->4)-alpha-D-glucosidic linkages in polysaccharides containing three or more (1->4)-alpha-linked D-glucose units. Amylase enzymes act on starch, glycogen and related polysaccharides and oligosaccharides in a random manner; reducing groups are liberated in the alpha-configuration. Other examples of amylase enzymes include: Beta-amylase (E.C. 3.2.1.2), Glucan 1,4-alpha-maltotetraohydrolase (E.C. 3.2.1.60), Isoamylase (E.C. 3.2.1.68), Glucan 1,4-alpha-maltohexaosidase (E.C. 3.2.1.98), and Glucan 1,4-alpha-maltohydrolase (E.C. 3.2.1.133).

Many amylase enzymes have been described in patents and published patent applications including, but not limited to: WO 2002/068589, WO 2002/068597, WO 2003/083054, WO 2004/091544, and WO 2008/080093.

Amylases are known to derived from Bacillus licheniformis having SEQ ID NO:2 as described in WO 95/10603. Suitable variants are those which are at least 90% identical to SEQ ID NO: 2 as described in WO 95/10603 and/or comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Such variants are described in WO 94/02597, WO 94/018314, WO 97/043424 and SEQ ID NO:4 of WO 99/019467.

Amylases are known to derived from B. stearothermophilus having SEQ ID NO:6 as described in WO 02/10355 or an amylase which is at least 90% identical thereto having amylolytic activity. Suitable variants of SEQ ID NO:6 include those which is at least 90% identical thereto and/or further comprise a deletion in positions 181 and/or 182 and/or a substitution in position 193. Amylases are known to derived from Bacillus sp. 707 having SEQ ID NO:6 as disclosed in WO 99/19467 or an amylase which is at least 90% identical thereto having amylolytic activity. Amylases are known from Bacillus halmapalus having SEQ ID NO:2 or SEQ ID NO:7 as described in WO 96/23872, also described as SP-722, or an amylase which is at least 90% identical to one of the sequences which has amylolytic activity.

Amylases are known to derived from Bacillus sp. DSM 12649 having SEQ ID NO:4 as disclosed in WO 00/22103 or an amylase which is at least 90% identical thereto having amylolytic activity. Amylases are known from Bacillus strain TS-23 having SEQ ID NO:2 as disclosed in WO 2009/061380 or an amylase which is at least 90% identical thereto having amylolytic activity. Amylases are known from Cytophaga sp. having SEQ ID NO:1 as disclosed in WO 2013/184577 or an amylase which is at least 90% identical thereto having amylolytic activity.

Amylases are known from Bacillus megaterium DSM 90 having SEQ ID NO:1 as disclosed in WO 2010/104675 or an amylase which is at least 90% identical thereto having amylolytic activity.

Amylases are known having amino acids 1 to 485 of SEQ ID NO:2 as described in WO 00/60060 or amylases comprising an amino acid sequence which is at least 96% identical with amino acids 1 to 485 of SEQ ID NO:2 which have amylolytic activity.

Amylases are also known having SEQ ID NO: 12 as described in WO 2006/002643 or amylases having at least 80% identity thereto and have amylolytic activity. Suitable amylases include those having at least 80% identity compared to SEQ ID NO:12 and/or comprising the substitutions at positions Y295F and M202LITV and have amylolytic activity.

Amylases are also known having SEQ ID NO:6 as described in WO 2011/098531 or amylases having at least 80% identity thereto having amylolytic activity. Suitable amylases include those having at least 80% identity compared to SEQ ID NO:6 and/or comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] and have amylolytic activity.

Amylases are known having SEQ ID NO:1 as described in WO 2013/001078 or amylases having at least 85% identity thereto having amylolytic activity. Suitable amylases include those having at least 85% identity compared to SEQ ID NO:1 and/or comprising an alteration at two or more (several) positions corresponding to positions G304, W140, W189, D134, E260, F262, W284, W347, W439, W469, G476, and G477 and having amylolytic activity.

Amylases are known having SEQ ID NO:2 as described in WO 2013/001087 or amylases having at least 85% identity thereto and having amylolytic activity. Suitable amylases include those having at least 85% identity compared to SEQ ID NO:2 and/or comprising a deletion of positions 181+182, or 182+183, or 183+184, which have amylolytic activity. Suitable amylases include those having at least 85% identity compared to SEQ ID NO:2 and/or comprising a deletion of positions 181+182, or 182+183, or 183+184, which comprise one or two or more modifications in any of positions corresponding to W140, W159, W167, Q169, W189, E194, N260, F262, W284, F289, G304, G305, R320, W347, W439, W469, G476 and G477 and have amylolytic activity.

Amylases also include hybrid α-amylase from above mentioned amylases as for example as described in WO 2006/066594.

Commercially available amylase enzymes include: Amplify®, Duramyl™, Termamyl™, Fungamyl™, Stainzyme™, Stainzyme Plus™, Natalase™, Liquozyme X and BAN™ (from Novozymes A/S), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont).

Lipase

“Lipases”, “lipolytic enzyme”, “lipid esterase”, all refer to an enzyme of EC class 3.1.1 (“carboxylic ester hydrolase”). Lipases (E.C. 3.1.1.3, Triacylglycerol lipase) may hydrolyze triglycerides to more hydrophilic mono- and diglycerides, free fatty acids, and glycerol. Lipase enzymes usually includes also enzymes which are active on substrates different from triglycerides or cleave specific fatty acids, such as Phospholipase A (E.C. 3.1.1.4), Galactolipase (E.C. 3.1.1.26), cutinase (EC 3.1.1.74), and enzymes having sterol esterase activity (EC 3.1.1.13) and/or wax-ester hydrolase activity (EC 3.1.1.50).

Many lipase enzymes have been described in patents and published patent applications including, but not limited to: WO2000032758, WO2003/089620, WO2005/032496, WO2005/086900, WO200600976, WO2006/031699, WO2008/036863, WO2011/046812, and WO2014059360.

Lipases are used in detergent and cleaning products to remove grease, fat, oil, and dairy stains. Commercially available lipases include but are not limited to: Lipolase™ Lipex™, Lipolex™ and Lipoclean™ (Novozymes A/S), Lumafast (originally from Genencor) and Lipomax (Gist-Brocades/now DSM).

The methods for determining lipolytic activity are well-known in the literature (see e.g. Gupta et al. (2003), Biotechnol. Appl. Biochem. 37, p. 63-71). E.g. the lipase activity may be measured by ester bond hydrolysis in the substrate para-nitrophenyl palmitate (pNP-Palmitate, C:16) and releases pNP which is yellow and can be detected at 405 nm.

Cellulase

“Cellulases”, “cellulase enzymes” or “cellulolytic enzymes” are enzymes involved in hydrolysis of cellulose. Three major types of cellulases are known, namely endo-ss-1,4-glucanase (endo-1,4-β-D-glucan 4-glucanohydrolase, E.C. 3.2.1.4; hydrolyzing β-1,4-glucosidic bonds in cellulose), cellobiohydrolase (1,4-P-D-glucan cellobiohydrolase, EC 3.2.1.91), and ss-glucosidase (EC 3.2.1.21).

Cellulase enzymes have been described in patents and published patent applications including, but not limited to: WO1997/025417, WO1998/024799, WO2003/068910, WO2005/003319, and WO2009020459.

Commercially available cellulase enzymes include are Celluzyme™, Endolase™, Carezyme™ Cellusoft™, Renozyme™, Celluclean™ (from Novozymes A/S), Ecostone™, Biotouch™, Econase™, Ecopulp™ (from AB Enzymes Finland), Clazinase™, and Puradax HA™, Genencor detergent cellulase L, IndiAge™ Neutra (from Genencor International Inc./DuPont), Revitalenz™ (2000 from DuPont), Primafast™ (DuPont) and KAC500™ (from Kao Corporation).

Cellulases according to the invention have “cellulolytic activity” or “cellulase activity”. Assays for measurement of cellulolytic activity are known to those skilled in the art. For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

Mannanase

Mannase (E.C. 3.2.1.78) enzymes hydrolyse internal β-1,4 bonds in mannose. Polymers. “Mannanase” may be an alkaline mannanase of Family 5 or 26. Mannanase enzymes are known to be derived from wild-type from Bacillus or Humicola, particularly B. agaradhaerens, B. licheniformis, B. halodurans, B. clausii, or H. insolens. Suitable mannanases are described in WO 99/064619.

Commercially available mannanase enzymes include: Mannaway® (Novozymes AIS).

Pectate Lyase

Pectate lyase (E.C. 4.2.2.2) enzymes eliminative cleavage of (1->4)-alpha-D-galacturonan to give oligosaccharides with 4-deoxy-alpha-D-galact-4-enuronosyl groups at their non-reducing ends.

Pectate lyase enzymes have been described in patents and published patent applications including, but not limited to: WO2004/090099. Pectate lyase are known to be derived from Bacillus, particularly B. licheniformis or B. agaradhaerens, or a variant derived of any of these, e.g. as described in U.S. Pat. No. 6,124,127, WO 99/027083, WO 99/027084, WO 2002/006442, WO 2002/092741, WO 2003/095638.

Commercially available pectate lyase enzymes include: Xpect™, Pectawash™ and Pectaway™ (Novozymes A/S); PrimaGreen™, EcoScour (DuPont).

Nuclease

Nuclease (EC 3.1.21.1) also known as Deoxyribonuclease I, or DNase preforms endonucleolytic cleavage to 5′-phosphodinucleotide and 5′-phosphooligonucleotide end-products. Nuclease enzymes have been described in patents and published patent applications including, but not limited to: U.S. Pat. No. 3,451,935, GB1300596, DE10304331, WO2015155350, WO2015155351, WO2015166075, WO2015181287, and WO2015181286.

A preferred embodiment of the present invention is a fermentation process for cultivating a Bacillus licheniformis cell in a chemically defined fermentation medium comprising the steps of
(a) providing a chemically defined fermentation medium,
(b) inoculating the fermentation medium of step (a) with a Bacillus licheniformis cell comprising a gene encoding an alkaline protease or an amylase under the control of an inducer-independent promoter, preferably an aprE promoter sequence,
(c) cultivating the Bacillus licheniformis cell in the fermentation medium under conditions conductive for the growth of the Bacillus licheniformis cell and the expression of the alkaline protease or the amylase,
   wherein the cultivation of the Bacillus licheniformis cell comprises the addition of one or more feed solutions comprising glucose and magnesium ions, and preferably trace elements, to the fermentation medium, and
   wherein the total amount of glucose added in the fermentation process is above 200 g of glucose per liter of initial fermentation medium; and
   wherein at least 0.1 gram, preferably 0.3-0.5 gram, magnesium ions per liter of initial fermentation medium is added to the fermentation medium during the cultivation of the Bacillus licheniformis cell by the one or more feed solutions comprising the magnesium ions; and
   wherein, preferably, the pH of the fermentation process is kept above 7.0, preferably, to pH 7.2 to pH 8.0, preferably by the addition of ammonium ions to the fermentation broth, and wherein, preferably, the fermentation is carried out under aerobic conditions for a duration of at least 24 hours, preferably at least 40 hours.

Downstream Processing

The protein of interest may or may not be further purified from the fermentation broth. Thus, in one embodiment, the present invention refers to a fermentation broth comprising a protein of interest obtained by a fermentation process as described herein.

In another embodiment, the protein of interest may be further purified from the fermentation broth. Thus, in one embodiment the present invention refers to a method of producing a protein of interest comprising a fermentation process described herein in further details comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium,
   wherein the total amount of chemically defined carbon source added in the fermentation process is above 200 g of carbon source per liter of initial fermentation medium;
   wherein at least 0.1 gram magnesium ions per liter of initial fermentation medium is added to the fermentation medium during the cultivation of the Bacillus cell by the one or more feed solutions comprising the magnesium ions; and
(d) purifying the protein of interest from the fermentation broth.

The desired protein may be secreted (into the liquid fraction of the fermentation broth) or may not be secreted from the host cells (and therefore is comprised in the cells of the fermentation broth). Depending on this, the desired protein may be recovered from the liquid fraction of the fermentation broth or from cell lysates. Recovery of the desired protein can be achieved by methods known to those skilled in the art. Suitable methods for recovery of proteins from fermentation broth include but are not limited to collection, centrifugation, filtration, extraction, and precipitation. If the protein of interest precipitates or crystallizes in the fermentation broth or binds at least in part to the particulate matter of the fermentation broth additional treatment steps might be needed to release the protein of interest from the biomass or to solubilize protein of interest crystals and precipitates. U.S. Pat. No. 6,316,240B1, WO2008110498A1, and WO2018185048A1 describe a method for recovering a protein of interest, which precipitates and/or crystallizes during fermentation, from the fermentation broth. Also WO2017097869A1 describes a method of purifying the protein of interest from a fermentation broth. In case the desired protein is comprised in the cells of the fermentation broth release of the protein of interest from the cells might be needed. Release from the cells can be achieved for instance, but not being limited thereto, by cell lysis with techniques well known to the skilled person, e.g., lysozyme treatment, ultrasonic treatment, French press or combinations thereof.

The protein of interest may be purified from the fermentation broth by methods known in the art. For example, a protein of interest may be isolated from the fermentation broth by conventional procedures including, but not limited to, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). The purified polypeptide may then be concentrated by procedures known in the art including, but not limited to, ultrafiltration and evaporation, in particular, thin film evaporation.

In another embodiment, the protein of interest is not purified from the fermentation broth. Thus, in one embodiment the present invention refers to a method of producing a protein of interest comprising a fermentation process described herein in further details comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium, and
   wherein the total amount of chemically defined carbon source added in the fermentation process is above 200 g of carbon source per liter of initial fermentation medium; and
   wherein at least 0.1 gram magnesium ions per liter of initial fermentation medium is added to the fermentation medium during the cultivation of the Bacillus cell by the one or more feed solutions comprising the magnesium ions.

Purifying a protein of interest from a fermentation broth is usually associated with residual components from the fermentation remaining in the purified protein solution. These remaining components are sometimes difficult to remove or can be removed with complex purification procedures. These contaminations can be the Bacillus cells or fractions thereof and/or products of the metabolism of the Bacillus cell, but often also medium components. The latter is in particular a problem with complex fermentation media as these types of media comprise a large variety of undefined compounds that often also interfere with the activity of the protein of interest, e.g., inhibiting enzyme activity. Using a chemically defined medium for industrial protein production overcomes this disadvantage, facilitates protein purification and leads to purified protein compositions free of interfering complex media components. Thus, in one embodiment, the present invention refers to a composition comprising a protein of interest produced by a method comprising the use of the fermentation process as described herein. Such compositions can be discriminated from compositions obtained with state of the art fermentation methods using complex media, because of the limited number or even by the absence of residual components resulting from the use of complex media. Preferably, the composition comprising a protein of interest obtained by the fermentation process of the present invention does not comprise components resulting from the use of complex media components.

Thus, in another embodiment, the present invention refers to a composition comprising a protein of interest produced by a method comprising the use of the fermentation process described herein in further details comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest, wherein the cultivation of the Bacillus cell comprises the addition of one or more feed solutions comprising one or more chemically defined carbon sources and magnesium ions to the fermentation medium, and wherein the total amount of chemically defined carbon source added in the fermentation process is above 200 g of carbon source per liter of initial fermentation medium; and wherein at least 0.1 gram magnesium ions per liter of initial fermentation medium is added to the fermentation medium during the cultivation of the Bacillus cell by the one or more feed solutions comprising the magnesium ions; and
(d) purifying the protein of interest from the fermentation broth and thereby forming the composition comprising the protein of interest.

In one embodiment, the protein of interest is not further purified. In this embodiment, the present invention refers to a composition comprising a protein of interest produced by a method comprising the fermentation process described herein in further details comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
   wherein the cultivation of the Bacillus cell comprises the addition of one or more feed solutions comprising one or more chemically defined carbon sources and magnesium ions to the fermentation medium, and
   wherein the total amount of chemically defined carbon source added in the fermentation process is above 200 g of carbon source per liter of initial fermentation medium; and
   wherein at least 0.1 gram magnesium ions per liter of initial fermentation medium is added to the fermentation medium during the cultivation of the Bacillus cell by the one or more feed solutions comprising the magnesium ions.

The purified protein solution may be further processed to form a “protein formulation”. “Protein formulation” means any non-complex formulation comprising a small number of ingredients, wherein the ingredients serve the purpose of stabilizing the proteins comprised in the protein formulation and/or the stabilization of the protein formulation itself. The term “protein stability” relates to the retention of proteins activity as a function of time during storage or operation. The term “protein formulation stability” relates to the maintenance of physical appearance of the protein formulation during storage or operation as well as the avoidance of microbial contamination during storage or operation.

A “protein formulation” is a composition which is meant to be formulated into a complex formulation which itself may be determined for final use. A “protein formulation” according to the invention is not a complex formulation comprising several components, wherein the components are formulated into the complex formulation to exert each individually a specific action in a final application. A complex formulation may be without being limited thereto a detergent formulation, wherein individual detergent components are formulated in amounts effective in the washing performance of the detergent formulation.

The protein formulation can be either solid or liquid. Protein formulations can be obtained by using techniques known in the art. For instance, without being limited thereto, solid enzyme formulations can be obtained by extrusion or granulation. Suitable extrusion and granulation techniques are known in the art and are described for instance in WO9419444A1 and WO9743482A1. “Liquid” in the context of enzyme formulation is related to the physical appearance at 20° C. and 101.3 kPa. Liquid protein formulations may comprise amounts of enzyme in the range of 0.1% to 40% by weight, or 0.5% to 30% by weight, or 1% to 25% by weight, or 3% to 10%, all relative to the total weight of the enzyme formulation.

The liquid protein formulation may comprise more than one type of protein. Aqueous protein formulations of the invention may comprise water in amounts of more than about 50% by weight, more than about 60% by weight, more than about 70% by weight, or more than about 80% by weight, all relative to the total weight of the protein formulation.

Protein formulations of the invention may comprise residual components such as salts originating from the fermentation medium, cell debris originating from the production host cells, metabolites produced by the production host cells during fermentation.

In one embodiment, residual components may be comprised in liquid enzyme formulations in amounts less than 30% by weight, less than 20% by weight less, than 10% by weight, or less than 5% by weight, all relative to the total weight of the aqueous protein formulation. In one embodiment, the protein formulation, in particular the liquid protein formulation, comprises in addition to the one or more protein one or more additional compounds selected from the group consisting of solvent, salt, pH regulator, preservative, stabilizer, enzyme inhibitors, chelators, and thickening agent. The preservative in a liquid protein formulation maybe a sorbitol, a benzoate, a proxel, or any combination therefore. The stabilizers in a liquid protein formulation maybe an MPG, a glycerol, an acetate, or any combination thereof. The chelators in a liquid protein formulation maybe a citrate. Enzyme inhibitors, in particular for proteases, may be boric acid, boronic acid derivatives, in particular phenyl boronic acid derivatives like 4FPBA, or peptide aldehydes. The protein as produced by the method of the present invention may be used in food, for example the protein can be an additive for baking. The protein can be used in feed, for example the protein is an animal feed additive. The protein can be used in the starch processing industry, for example amylases are used in the conversion of starch to ethanol or sugars (high fructose corn syrup) and other byproducts such as oil, dry distiller's grains, etc. The protein maybe used in pulp and paper processing, for example, the protein can be used for improving paper strength. In one embodiment, the protein produced by the methods of the present invention are used in detergent formulations or cleaning formulations. “Detergent formulation” or “cleaning formulation” means compositions designated for cleaning soiled material. Cleaning includes laundering and hard surface cleaning. Soiled material according to the invention includes textiles and/or hard surfaces.

The invention is further illustrated in the following examples which are not intended to be in any way limiting to the scope of the invention as claimed.

### EXAMPLES

The following examples only serve to illustrate the invention. The numerous possible variations that are obvious to a person skilled in the art also fall within the scope of the invention.

Unless otherwise stated the following experiments have been performed by applying standard equipment, methods, chemicals, and biochemicals as used in genetic engineering and fermentative production of chemical compounds by cultivation of microorganisms. See also Sambrook et al. (Molecular Cloning: A Laboratory Manual. 2nd edition, Cold Spring Harbor Laboratory, Cold Spring Harbor Laboratory Press, Cold Spring Harbor, N.Y., 1989) and Chmiel et al. (Bioprocesstechnik 1. Einführung in die Bioverfahrenstechnik, Gustav Fischer Verlag, Stuttgart, 1991).

### Example 1

Bacillus Strain

Bacillus licheniformis ATCC53926 cell comprising a gene encoding an alkaline protease as described in WO9102792.

The expression of the alkaline protease was under the control aprE promoter from Bacillus licheniformis ATCC 53926 as described in WO9102792. The alkaline protease expressed was the alkaline protease from Bacillus lentus (BLAP) as specified in WO9102792 comprising the mutation R99E.

Fermentation Conditions

Bacillus licheniformis cell was inoculated in a chemically defined fermentation medium containing the components listed in Table 1 and Table 2.

TABLE 1
Composition of initial fermentation medium.

| Compound | Formula | Concentration [g/L] |
|---|---|---|
| Citric acid | C6H8O7 | 3.0 |
| Calcium sulphate | CaSO4 | 0.7 |
| Monopotassium phosphate | KH2PO4 | 25 |
| Trace element solution | (Table 2) | 18 |
| Magnesium sulfate | MgSO4 | 0.5 |
| Sodium hydroxide | NaOH | 4.0 |
| Ammonia | NH3 | 1.3 |

TABLE 2
Trace element composition of the trace element
solution comprising 40 g/L citric acid.

| Trace element | Symbol | Concentration [mM] |
|---|---|---|
| Manganese | Mn | 24 |
| Zinc | Zn | 17 |
| Copper | Cu | 32 |
| Cobalt | Co | 1 |
| Nickel | Ni | 2 |
| Molybdenum | Mo | 0.2 |
| Iron | Fe | 38 |

A solution containing glucose and magnesium ions was used as feed solution. The amount of magnesium ions added via the feed solution resulted in a total of 0.4 g magnesium ions per liter of initial fermentation medium.

A control fermentation was performed under the same conditions, but the amount of magnesium that was supplied as feed solution in the first experiment was now supplied additionally in the initial fermentation medium. The feed solution of the control experiment did not contain magnesium. In both experiments, the total amount of added chemically defined carbon source was kept above 200 g per liter of initial medium in accordance to the requirements of industrially relevant fermentation processes. pH of the fermentation processes was kept above 7 by addition of ammonium ions to the fermentation broth. Fermentations were carried out under aerobic conditions for a duration of above 48 hours.

Measurement of Protease Titer

The titer of the produced protease for the fermentation process was determined at various time points. Proteolytic activity was determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring OD405.

Result

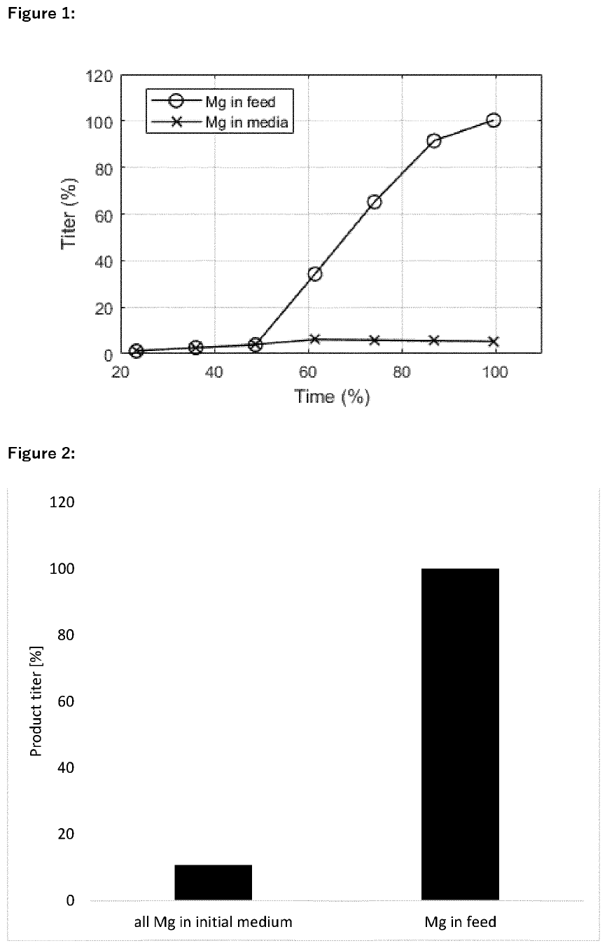

FIG. 1 shows the development of the protease titer over time. From FIG. 1 it can be derived that after a certain fermentation time, the protease titer increases with a higher slope when magnesium ions are added as a feed solution. FIG. 2 shows the protease titer at the end of the fermentation process. From FIG. 2 it can be derived that the achieved titer of the produced protease in the fermentation process with magnesium in feed was found to be over ten times higher than the control. Thus, adding the same amount of magnesium ions to the fermentation process as a feed solution and not to the batch medium increased the amount of protein of interest produced by the Bacillus cell.

### Example 2

Extraction and alignment of Bacillus species promoters

A translated blast search using tblastn 2.5.0+ (Camacho C., Coulouris G., Avagyan V., Ma N., Papadopoulos J., Bealer K., & Madden T.L. (2008) “BLAST+: architecture and applications.” BMC Bioinformatics 10:421) was performed using aprE protein sequence from Bacillus licheniformis (SEQ ID NO. 2) as a query against Genbank and Genbank WGS (Whole Genome Shotgun) databases, with options: -evalue 1e-20, -db_gencode 11, -max_target_seqs 60000. Full GenBank records were retrieved for BLAST hits above minimal protein identity of 40%.

Using BLAST hit location information from the blast search results, upstream sequences of aprE-coding genes were extracted, subject to the following conditions:
a. Upstream extraction size was 200 nucleotides. If there was an upstream gene/CDS annotation closer than 200 nucleotides, then a shorter fragment was extracted. If fragment length was less than 50 nucleotides, such a fragment was not extracted.
b. Extracted upstream sequences were grouped by BLAST hit bitscore, and sorted in descending order by the same bitscore. To avoid bias, identical upstream sequences from the same bitscore group were deduplicated.
c. For each of by-bitscore upstream sequence groups, a cumulative multiple alignment was performed (and saved separately) using mafft version 7.307 (Katoh, Standley. “MAFFT multiple sequence alignment software version 7: improvements in performance and usability”, Molecular Biology and Evolution 30:772-780, 2013), with the keeplength option. Generated multiple nucleotide alignments were visualized as sequence logos and examined to identify the bitscore threshold at which upstream regulatory sequences conservation is the most apparent: conserved fragments still have high information content, while non-conserved fragments have low information content.
d. Based on the identified threshold, all the upstream sequences with bitscore above the threshold (SEQ ID No. 19 to 166) were multiple-aligned using mafft.

Hidden Markov Model (HMM) Creation

Using the above created multiple alignment file, an hmm was build using HMMER 3.1b1 (Wheeler, Travis J, and Sean R Eddy. (2013) “nhmmer: DNA homology search with profile HMMs.” Bioinformatics (Oxford, England) vol. 29, 19 (2013): 2487-9), by running the command: hmmbuild -n PaprE PaprE.hmm {aligned.mfa}. This hmm was then pressed using: hmmpress PaprE.hmm, resulting in a model that can be run over any sequence.

Sequence Extraction

In order to extract the sequence matching the model, the HMMER software can be run using the command: nhmmscan PaprE.hmm {sequence}, where {sequence} represents a fasta formatted file containing any DNA sequence. This will output a list of sequences matching the model (given by start and end of the match), together with an e-value and a score. Calibration of the hmm indicated that any score above a cutoff of 50 is indicative of a match. Using this cutoff to extract matching sequences from a database of over 8000 non-Bacilli genomes, a false discovery rate of zero was confirmed.

1. A fermentation process for cultivating a Bacillus cell in a chemically defined fermentation medium comprising the steps of
(a) providing a chemically defined fermentation medium,
(b) inoculating the fermentation medium of step (a) with a Bacillus cell comprising a gene encoding a protein of interest under the control of an inducer-independent promoter,
(c) cultivating the Bacillus cell in the fermentation medium under conditions conductive for the growth of the Bacillus cell and the expression of the protein of interest,
wherein the cultivation of the Bacillus cell comprises the addition of one or more feed solutions comprising one or more chemically defined carbon sources and magnesium ions to the fermentation medium, and
wherein the total amount of chemically defined carbon source added in the fermentation process is above 200 g of carbon source per liter of initial fermentation medium; and
wherein at least 0.1 gram magnesium ions per liter of initial fermentation medium is added to the fermentation medium during the cultivation of the Bacillus cell by the one or more feed solutions comprising the magnesium ions.

2. The fermentation process of claim 1, wherein the Bacillus cell has not been genetically modified in its ability to take up or metabolize an inducer molecule.

3. The fermentation process of claim 1, wherein the expression of the gene of interest is under the control of a promoter sequence selected from the group consisting of an veg promoter, lepA promoter, serA promoter, ymdA promoter, fba promoter, aprE promoter, amyQ promoter, amyL promoter, bacteriophage SPO1 promoter, cryIIIA promoter, combinations thereof, and active fragments or variants thereof.

4. The fermentation process of claim 24, wherein the aprE promoter sequence has an HMM-score above 50.

5. The fermentation process of claim 24, or wherein the aprE promoter is selected from the group of aprE promoters from Bacillus amyloliquefaciens, Bacillus clausii, Bacillus haloduans, Bacillus lentus, Bacillus licheniformis, Bacillus pumilus, Bacillus subtilis, and Bacillus velezensis.

6. The fermentation process of claim 24, wherein the aprE promoter sequence is the promoter of the gene coding for the subtilisin Carlsberg protease or a functional fragment of the aprE promoter sequence or a functional variant of the aprE promoter sequence of the gene coding for the subtilisin Carlsberg protease, wherein the subtilisin Carlsberg protease has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 2, SEQ ID NO: 4, or SEQ ID NO: 6.

7. The fermentation process of claim 24, wherein the aprE promoter sequence comprises the sigma factor A core promoter.

8. The fermentation process of claim 24, wherein the aprE promoter sequence comprises one or more of the binding motifs of regulatory factors selected from the group consisting of degU (sacU), ScoC (hpr), SinR and AbrB.

9. The fermentation process of claim 24, wherein the aprE promoter sequence has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or even 100% sequence identity with SEQ ID NO: 8, SEQ ID NO: 10, SEQ ID NO: 12 or SEQ 13.

10. The fermentation process of claim 1, wherein 0.1-10 gram magnesium ions per liter of initial fermentation medium is added to the fermentation medium during the cultivation of the Bacillus cell by the one or more feed solutions comprising the magnesium ions.

11. The fermentation process of claim 1, wherein the magnesium ions are provided by one or more magnesium salts or by magnesium hydroxide or by combinations of one or more magnesium salts and magnesium hydroxide.

12. The fermentation process of claim 1, wherein one or more trace element ions are added to the fermentation medium during the cultivation of the Bacillus cell by one or more feed solutions comprising one or more trace element ions and the trace element ions are added during the cultivation of the Bacillus cell in an amount selected from the group consisting of at least 50 μmol per liter of initial medium iron, at least 40 μmol per liter of initial medium copper, at least 30 μmol per liter of initial medium manganese, and at least 40 μmol per liter of initial medium zinc.

13. The fermentation process of claim 12, wherein the one or more trace element ions added to the fermentation medium during cultivation of the Bacillus cell by the one or more feed solutions comprising one or more trace element ions further comprises one or more trace element ions selected from the group consisting of at least 1 μmol per liter of initial medium cobalt, at least 2 μmol per liter of initial medium nickel, and at least 0.3 μmol per liter of initial medium molybdenum.

14. The fermentation process of claim 1, wherein the chemically defined carbon source comprises glucose.

15. The fermentation process of claim 1, wherein one or more chemical defined nutrient sources selected from the group consisting of a chemically defined nitrogen source, chemically defined sulfur source and chemically defined potassium source are added to the fermentation medium during the cultivation of the Bacillus cell by one or more feed solutions comprising these nutrient sources.

16. The fermentation process of claim 1, wherein the pH of the fermentation broth during cultivation of the Bacillus cell is adjusted at or above pH 6.0, pH 6.5, pH 7.0, pH 7.2, pH 7.4, or pH 7.6.

17. The fermentation process of claim 1, wherein the fermentation process provides a titer of at least 5 g/l of protein of interest.

18. The fermentation process of claim 1, wherein the protein of interest is an enzyme.

19. The fermentation process of claim 1, wherein the fermentation product is secreted by the Bacillus cell into the fermentation broth.

20. A method of producing a protein of interest comprising the fermentation process of claim 1, and optionally purifying the protein of interest.

21. A fermentation broth comprising a protein of interest obtained by a fermentation process of claim 1.

22. A composition comprising a protein of interest produced by a method of claim 20.

23. A method for increasing the titer of a protein of interest above 5 g/L comprising the fermentation process of claim 1.

24. The fermentation process of claim 3, wherein the expression of the gene of interest is under the control of an aprE promoter sequence.
